(19) United States
(12) Patent Application Publication
(10) Pub. No.: US 2026/0337270 A1
(43) Pub. Date: Oct. 29, 2026

(54) COMPOSITIONS AND METHODS FOR TARGETING TRBJ2-3 T CELLS FOR TREATMENT OF RHEUMATOID ARTHRITIS

(71) Applicant: New York Society for the Relief of the Ruptured and Crippled, Maintaining the Hospital for Special S, New York, NY (US)

(72) Inventors: Amit Lakhanpal, New York, NY (US); Laura Donlin, New York, NY (US)

(21) Appl. No.: 19/393,266

(22) Filed: Nov. 18, 2025

Related U.S. Application Data
(60) Provisional application No. 63/721,672, filed on Nov. 18, 2024.

(51) Int. Cl. C07K 16/28; A61P 19/02; C12N 15/113

(52) U.S. Cl. CPC C07K 16/2809; A61P 19/02; C12N 15/1138; C12N 2310/11; C12N 2310/14; C12N 2310/141

(57) ABSTRACT

Provided herein are methods of treatment Rheumatoid Arthritis (RA) using compounds that deplete CD4+ T cells comprising TRBJ2-3.

### CROSS-REFERENCE TO RELATED APPLICATIONS

This application claims the benefit of U.S. Provisional Patent Application No. 63/721,672, filed Nov. 18, 2024. This application is incorporated by reference in its entirety.

### BACKGROUND OF THE INVENTION

The adaptive immune system consists of T cells with unique receptors that collectively can recognize an extraordinary range of diverse antigens yet must also productively interact with Human Leukocyte Antigen (HLA) complexes. To mediate these interactions, anchor points on HLA/antigen complexes interface with conserved patterns in the T cell receptor (TCR) [23-25]. For each HLA gene and their variant alleles, these anchor points are unique and may promote interactions with distinct TCR patterns, preferentially selecting a subset of T cells during development that then comprise the circulating T cell repertoire. Here we ask how HLA variants that associate with disease risk impact patterns of T cells across the TCR repertoire, focusing on the HLA-DRB1 alleles associated with rheumatoid arthritis (RA).

Rheumatoid arthritis (RA) is an autoimmune inflammatory disease in which damage and disability are caused by the immune system inappropriately attacking the joints. Existing therapies that suppress the immune system by various mechanisms are effective in some patients, but there still remain patients in whom existing therapies are insufficient to control their disease or cause intolerable side effects.

RA is hallmarked by a pronounced accumulation of immune cells in synovial tissue that can lead to joint damage and disability, with a global prevalence at ~300/100,000 [1, 2]. While multiple factors contribute to RA pathogenesis, the strongest genetic association lies in the MHC Class II subunit HLADRB1 gene [3]. This association has been finely mapped to a small number of amino acids in HLA-DRB1 that bind to peptide antigens and interface with TCRs [4]. However, the mechanism underlying how specific HLA-DRB1 variants influence the risk of developing RA remains unclear.

Most studies related to HLA-DRB1 disease-associated amino acids have focused on how they impact binding of specific peptides and therein activate specific T cell clones. While a critical direction, to date evidence of a singular pathogenic T cell antigen or of unique pathogenic TCR clones mediating pathology is lacking, opening the possibility that broader patterns of TCR clones may be collectively involved in disease pathogenesis. This is particularly true for CD4 T cells, which relative to CD8 T cells, are found as smaller clonal populations in affected joints [26]. Thus, in this study we focused on whether diversity in HLA-DRB1 alleles impacts CD4 T cells on a compartment-wide scale to understand if disease risk may involve large numbers of TCR clones that share an identifiable pattern.

A single T cell clonotype can be defined by its unique TCR composition. Each TCR consists of a heterodimer, with each monomer generated by the recombination of various germline-encoded gene subunits, known as V, D and J genes. The TCR β monomer, for example, can be generated from one each of 45 functional TRBV, 2 TRBD, and 13 TRBJ genes. The TCR is further diversified by nucleotide insertions and deletions in the complementarity-determining region 3 (CDR3), defined conventionally as the segment between the distal V gene cysteine and the proximal J gene phenylalanine [27-29]. While the extensive diversity across TCRs permits identification of unique antigens, certain variable features have also been suggested to impact HLA interactions. For example, in large cohort studies comparing TCR repertoires from individuals with divergent HLA alleles, specific TCR V genes have been found to associate with discreet HLA amino acid positions [30-32]. Further, recently features of the TCR CDR3 region have been associated with HLA Class II amino acid variation that confer risk of autoimmune diseases [8]. These studies suggest that HLA variation influences the frequencies of TCR features, both germline-encoded and otherwise, in the circulating T cell repertoire.

A continuing need in the art exists for compositions and methods for effective and targeted treatment of RA.

### SUMMARY OF THE INVENTION

In one aspect, provided herein is a method for treating rheumatoid arthritis (RA), the method comprising administering to the subject a therapeutically effective amount of a compound that depletes CD4 positive (+) T cells comprising T cell receptor beta joining 2-3 (TRBJ2-3), T cell receptor beta variable 19 (TRBV19), T cell receptor beta variable 20-1 (TRBV20-1), or T cell receptor beta variable 15-1 (TRBV2-1). In certain embodiments, the compound depletes CD4+ T cells comprising TRBJ2-3. In certain embodiments, the compound is an immunoglobulin, optionally an antibody or an antibody fragment, optionally a monoclonal antibody, optionally a humanized monoclonal antibody. In certain embodiments, the compound is an immunoglobulin that targets TRBJ2-3. In certain embodiments, the compound is an immunoglobulin that targets C-terminal aspartic acid (D) amino acid of a complementarity-determining region 3 (CDR3) of the T cell receptor of the T cell. In certain embodiments, the compound is administered systemically, optionally intravenously. In certain embodiments, the compound is administered via a synovial injection. In certain embodiments, the compound is an antisense oligonucleotide (ASO), RNAi, small interfering RNA (siRNA), or microRNA (miRNA).

These and other aspects of the invention are apparent from the following detailed description of the invention.

### BRIEF DESCRIPTION OF THE FIGURES

FIG. 1A shows rheumatoid arthritis (RA) is a systemic autoimmune inflammatory disease characterized by immune-mediated joint damage. (Mayo Foundation for Medical Education and Research). FIG. 1B depicts the HLA-DRB1 gene is the strongest genetic association with RA. (Kochi et al., Nat Gen (2010) and Raychaudhuri et al., Nat Gen (2012)).

FIG. 2 demonstrates four residues in the HLA-DRB1 gene account for most of the association with RA. (Raychaudhuri et al., Nat Gen (2012)).

FIG. 3 shows TCR repertoire diversity derives from germline-encoded and randomized features. There are 10¹⁵ possible TCR sequences. (Davis and Bjorkman, Nature, 334(395), (August 1988) and Laydon et al, Phil Trans R Soc B., 370(1365), (August 2015)).

FIG. 4 shows specific DRB1* alleles increase the risk of RA.

FIG. 5A demonstrates HLA-DRB1 presents antigen to CD4 T cells in the periphery. FIG. 5B demonstrates HLA-DRB1 also presents antigen to developing CD4 T cells in the thymus.

HLA-DRB1 alleles select specific TCR features in the T cell repertoire.

FIG. 6 shows these HLA-DRB1 functions are candidates for the HLA-RA relationship mechanism.

FIG. 7 demonstrates HLA-DRB1* selects specific germline-encoded TCR sequences, specifically TRBJ2-3. TRBJ2-3 CD4 T cells are selected by HLA-DRB1* alleles.

FIG. 8 demonstrates TRBJ2-3 CD4 T cell clonotypes have a distal D(aa) selected by HLA-DRB1 *. Specific CDR3 features are selected by HLA-DRB1* including C-terminal D(aa). (Ishigaki et al. Nat Gen, 4(393)(2022)).

FIG. 9 shows TRBJ2-3 CD4 T cells have higher TCR-CDR3^{DRB1*} scores.

FIG. 10 shows that CDR3 region overlaps with the V and J genes proximally and distally. FIG. 11 shows TRBJ2-3 in CD4 T cells contributes a C-terminal D amino acid in CDR3,which is selected by HLA-DRB1*.

FIG. 12A-C demonstrates, in RA patients, TRBJ2-3 CD4 T cells most strongly reflect the DRB1* selection signature. (A) Our panel of seropositive RA patients is similar to typical baseline RA demographics. (B) The selection signature of HLA-DRB1* is also found in RA subjects across the entire CD4 T cell repertoire. (C) In RA patients, the repertoire-wide TCR-CDR3^{DRB1*} selection signature is largely due to the TRBJ2-3 CD4 T cells.

FIG. 13 shows TRBJ2-3 CD4 T cell frequencies are higher in synovium than in blood.

FIG. 14A shows that the relationship between mean CDR3 risk score (reflecting the presence of TCRβ CDR3 features selected by HLA-DRB1) and the burden of RA-associated HLA-DRB1 alleles (quantified as the RA HLA risk score) across 19 RA patients shows an increasing abundance of those TCR features with increasing risky HLA allele burden.

FIG. 14B shows that the subset of CD4 T cells with the TRBJ2_3 rearrangement has a markedly higher enrichment of clones with features across all RA patients.

FIG. 14C shows that the subset of CD4 T cells with the TRBJ2_3 rearrangement has a markedly higher enrichment of clones with features as observed in each RA patient individually as well.

FIG. 14D shows that the frequency of CD4 T cell clones with TRBJ2_3 is higher in RA synovium than blood, uniquely across all TRBJ rearrangements.

FIG. 15 shows that individuals with RA risk HLA-DRB1 alleles have more CD4 T cells with a J2-3 encoded TCR. (A) Bulk blood TCRseq data and HLA-DRB1 typing were obtained from a large previously characterized cohort of healthy subjects. Plots in (B) and (C) show the proportion of the CD4 repertoire with the indicated V or J gene rearrangement versus the HLA-DRB1 RA risk score for each of the healthy subjects, with the OLS fit and p-value indicated.

Significant associations indicate selection by HLA-DRB1 alleles.

FIG. 16 shows that J2-3 encoded TCRs have CDR3 features preferentially selected by HLA-DRB1 RA risk alleles. (A) Using the association between TCRβ CDR3 features and HLA-DRB1-mediated risk for RA (motif derived from previous studies), the CDR3DRB1* risk score was computed for each cell for which single-cell TCR sequencing data was obtained. (B) Among CD4 T cell clones from the blood of RA patients, the CDR3DRB1* selection score is markedly higher in the subset characterized by the TRBJ2-3 rearrangement, suggesting that this subset is particularly enriched in features that are selected by HLA-DRB1 alleles that predispose to RA.

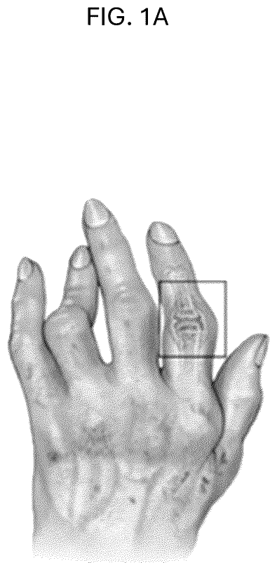

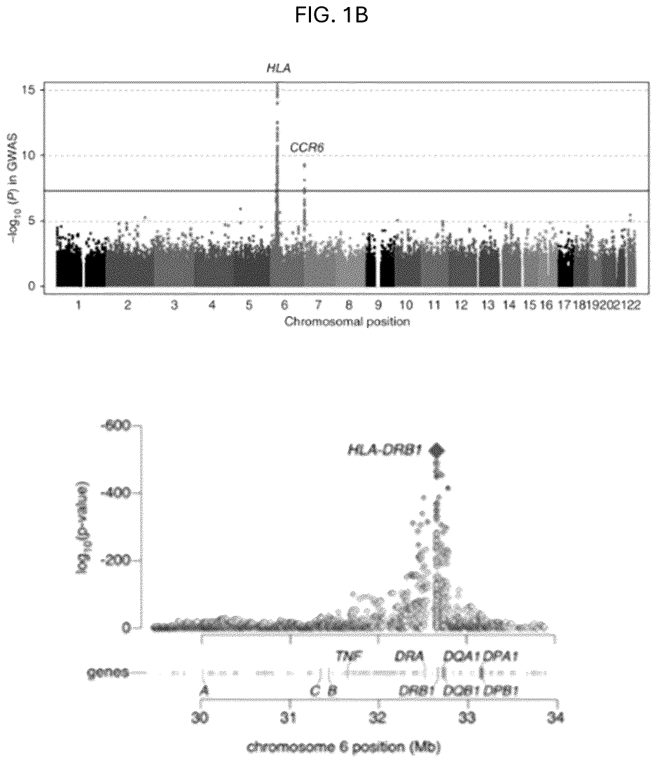

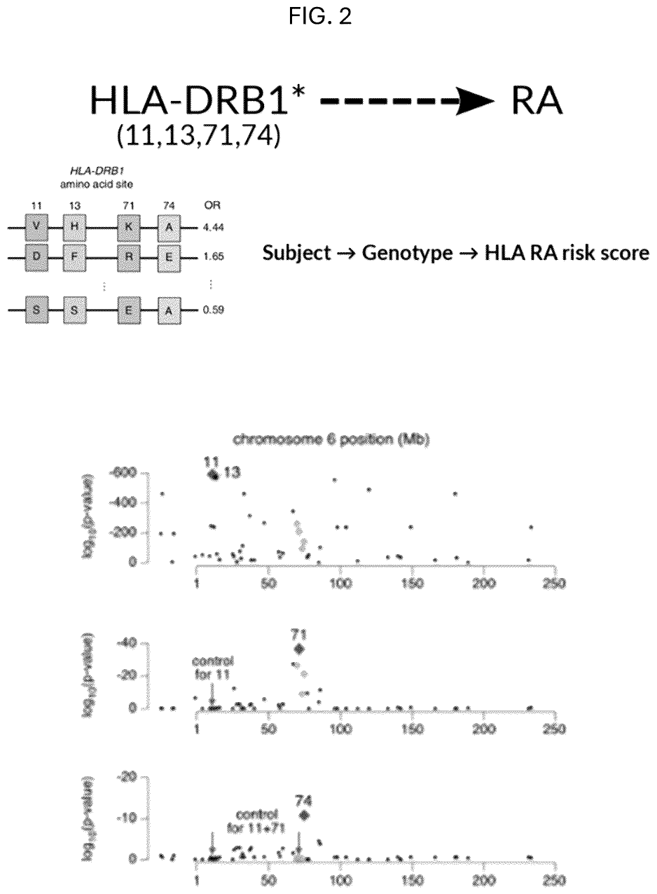

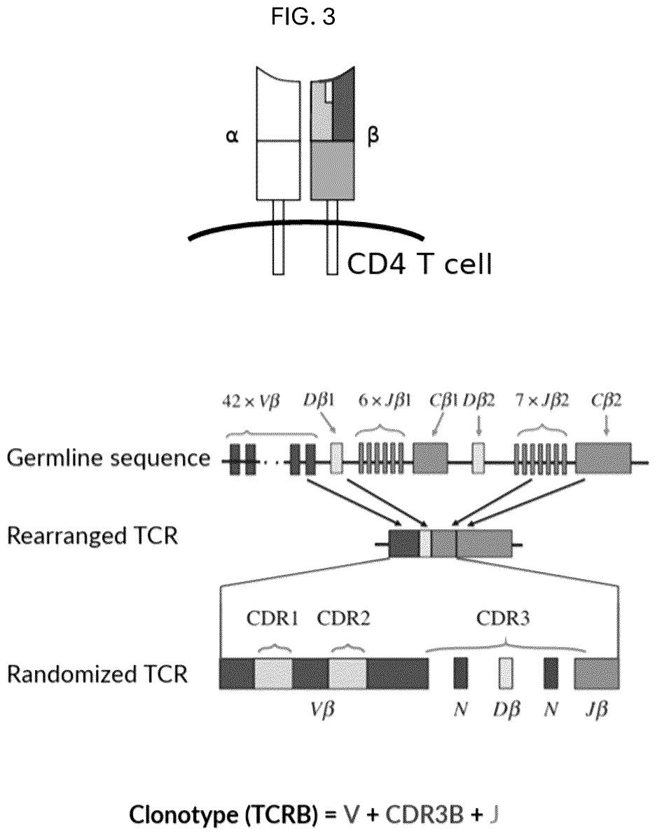

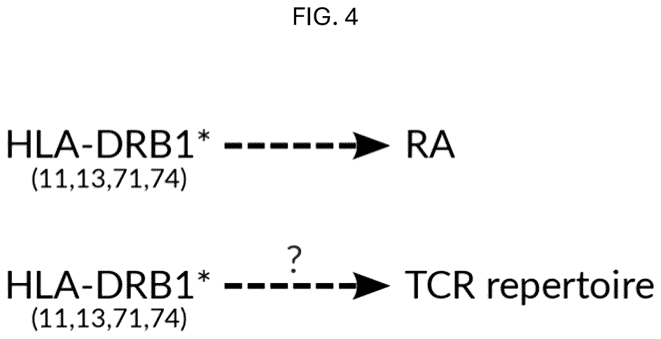

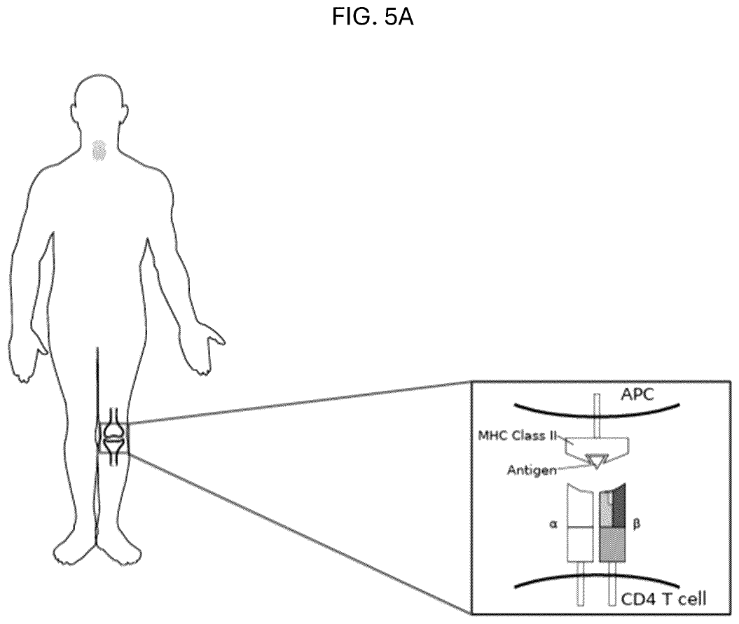

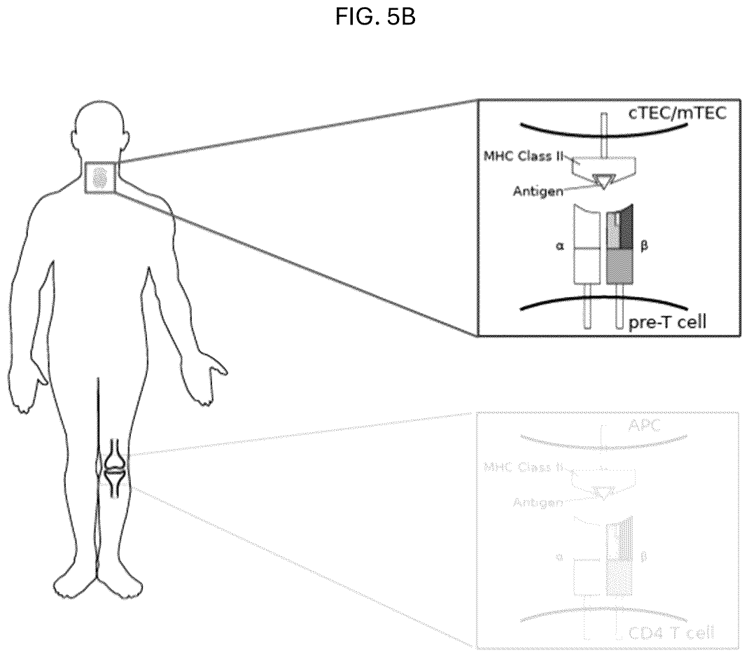

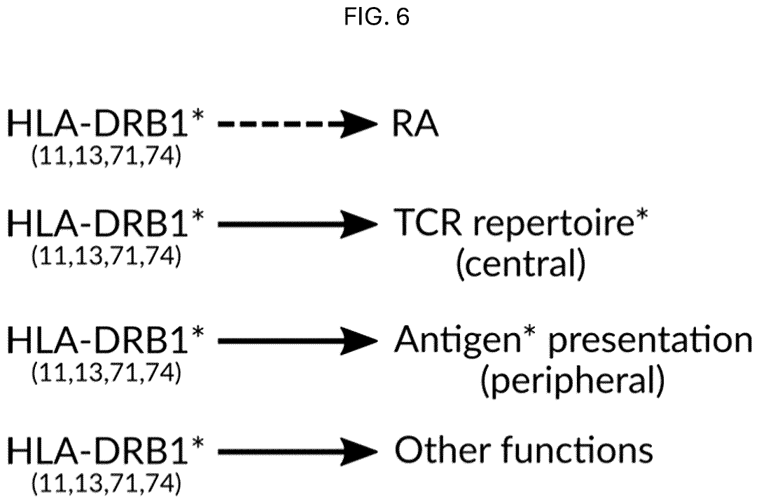

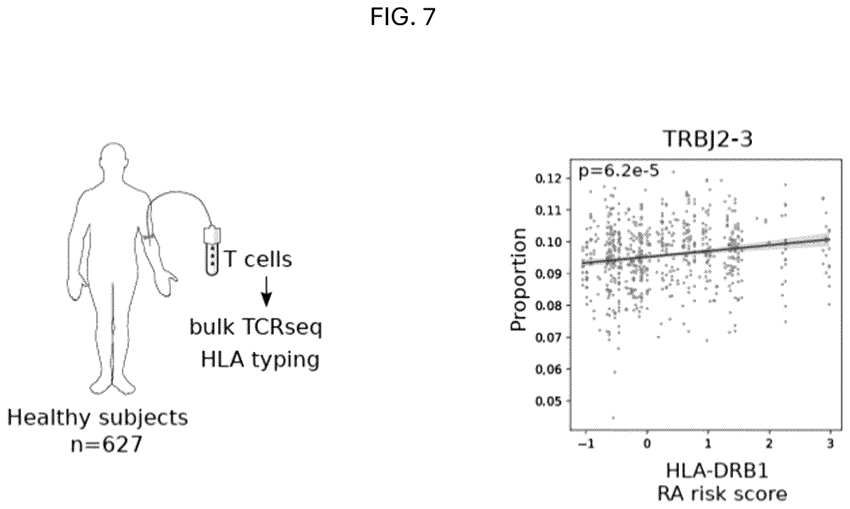

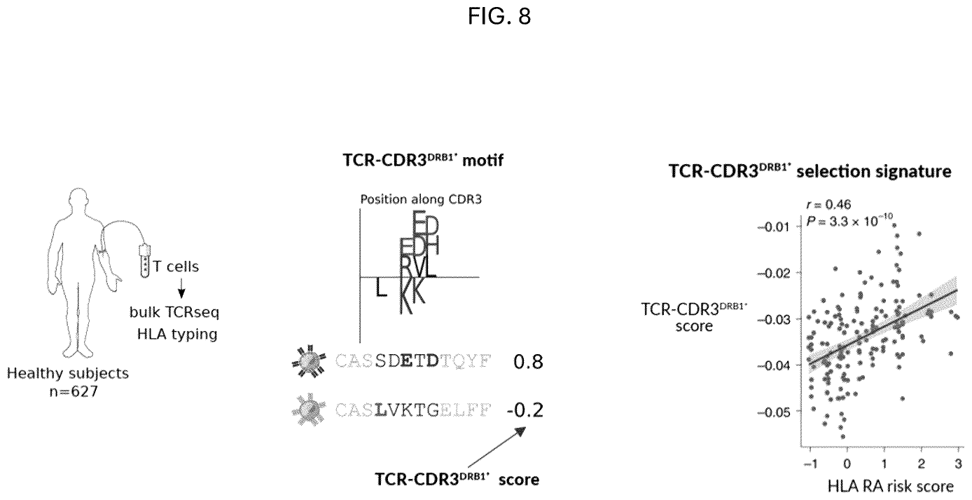

(C) For CD4 clones with a specific CDR3 length (12 to left, 13 to right), stratified by TRBJ gene rearrangement (rows), the identity (single-letter amino acid code) and proportion (blue color scale) of clones in each stratum with the most-frequent amino acid at each position (columns).

The dark blue boxes in the distal CDR3 region represent amino acids contributed by the germline-encoded TRBJ gene. Beneath each length-specific plot is a motif diagram reflecting the degree of germline-independent association of amino acids at each position with high-risk HLA-DRB1 alleles, from which those HLA selected amino acids that are predominant within a single TRBJ-rearranged subset are highlighted by green (for negative selection) or red (for positive selection) boxes. The presence of an HLA-DRB1-selected amino acid overlapping with the TRBJ gene-contributed sequence is unique to TRBJ2-3.

FIG. 17 shows that HLA-DRB1 alleles shape the CD4 TCR repertoire in RA patients. (A) Seventeen donors with RA provided blood samples, comprising approximately 6,500 cells per sample. (B) (upper) The repertoire-wide mean CD4 CDR3DRB1* score for CD4 T cells from RA patient blood samples. (lower) HLA-DRB1 RA risk score for each subject, with gray bars reflecting the combined effect of both HLA-DRB1 alleles on the genetic risk for RA, and the white bars representing the contribution from each allele separately. Patients are sorted first by the magnitude of their single highest-RA-risk HLA-DRB1 allele (tallest white bar), and second by the total HLA-DRB1 RA risk (grey bar). (C) The relationship between the mean CDR3DRB1* score and the HLA-DRB1 RA risk for each of the 26 samples across the 17 RA subjects.

FIG. 18 shows that HLA-DRB1 variants select specific CD4 T cells with J2-3 encoded TCRs in RA. The relationship between CDR3DRB1* selection score and HLA-DRB1-related RA risk is particularly strong in the subset of CD4 T cells with the TRBJ2-3 rearrangement, by contrast with the subset with the TRBJ2-6 rearrangement.

FIG. 19 shows that increased frequencies of CD4 T cells with TRBJ2-3 TCRs in RA joints.

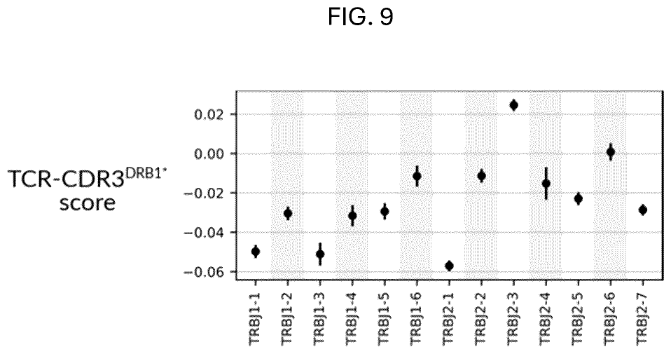

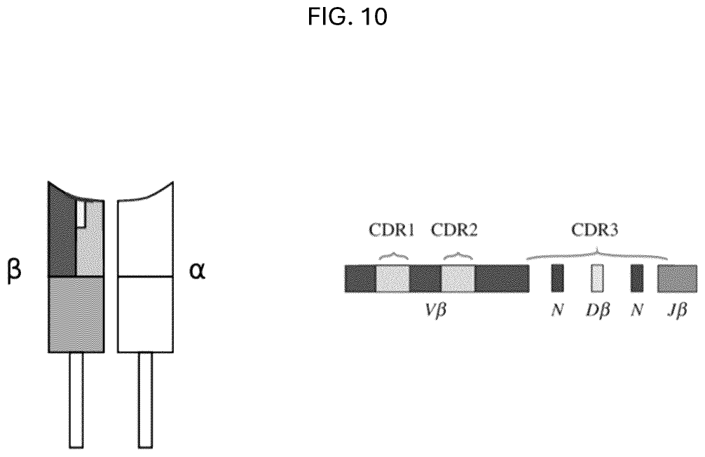

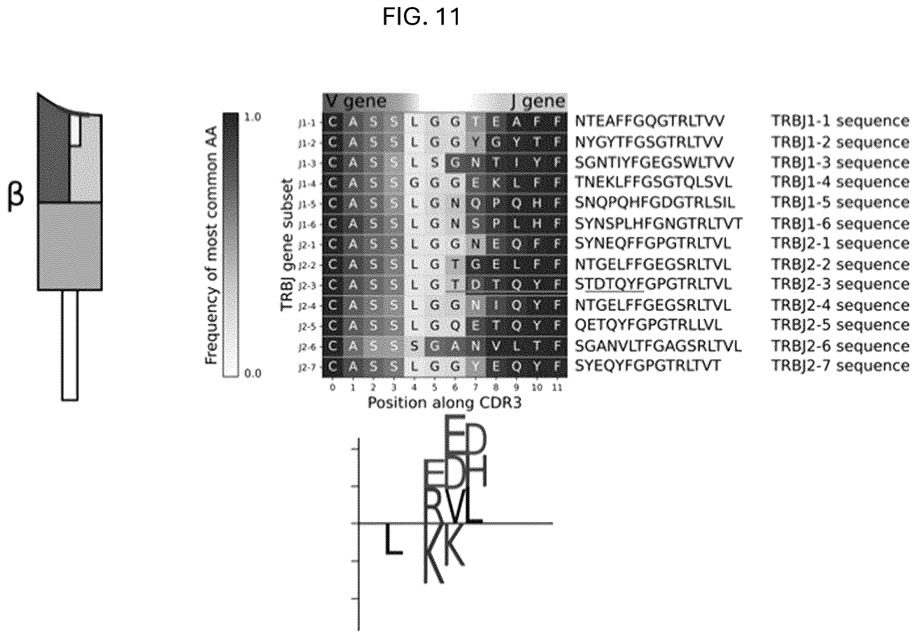

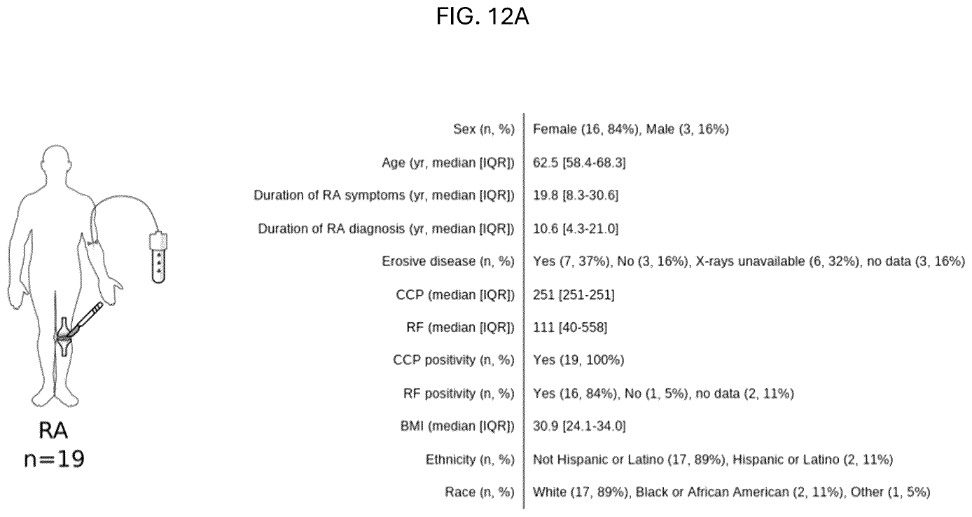

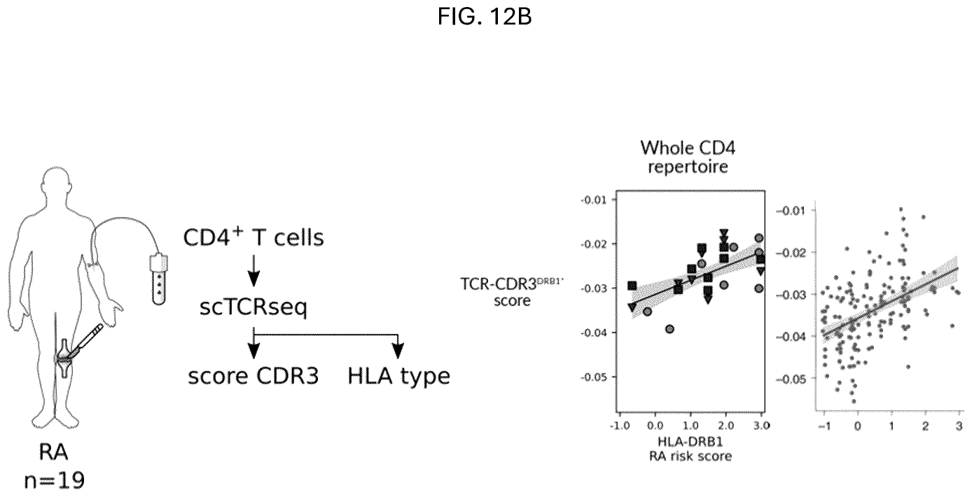

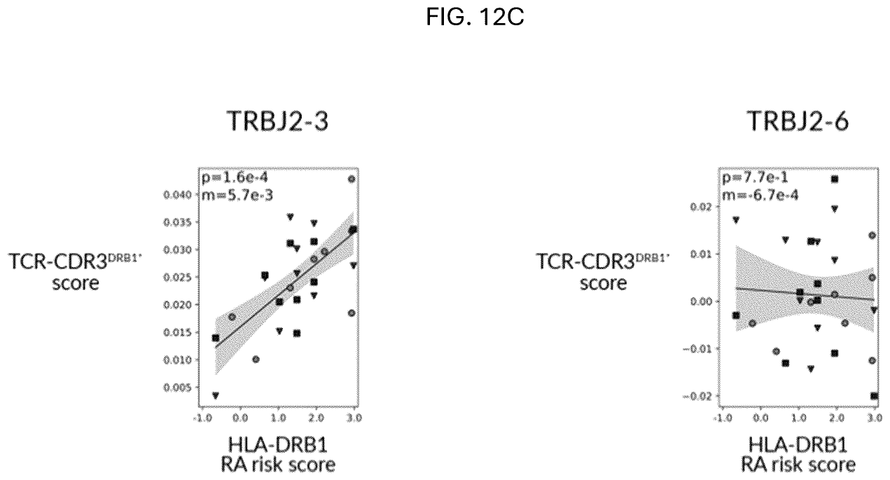

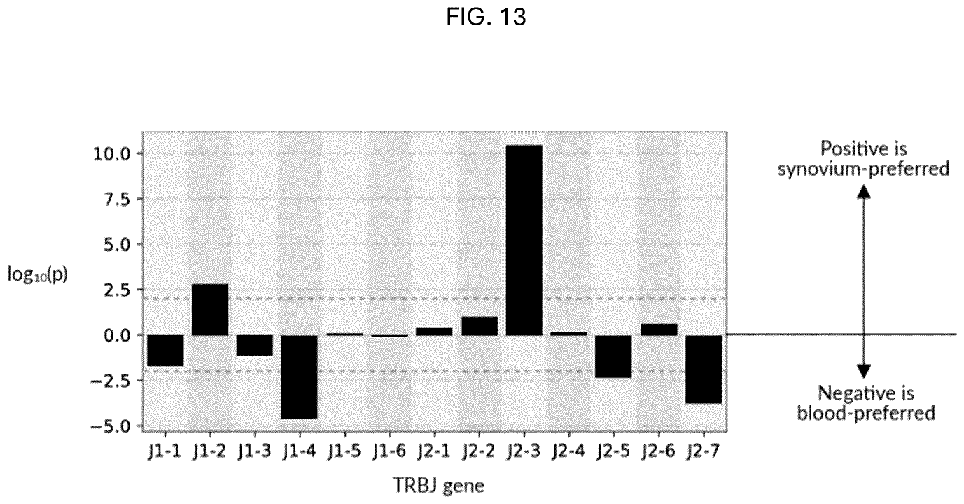

FIG. 20 shows basic quality control parameters for a single subject. “n_genes_by_counts” is the number of genes observed more than once in each cell, “total_counts” is the total number of counts observed in each cell, and “pct_counts_mt” is the percentage of total counts that originate from mitochondrial genes in each cell. The diagonals are histograms of each QC parameter's distribution, the below-diagonal plots are simple scatter plots for each pair of basic QC values, and the above-diagonal are hexagonal two-dimensional histograms for each pair. To achieve this in the relatively homogeneous CD4 T cell background population we adapted and independently implemented an optimized variant (FIG. 9) of the synthetic doublet methods.

FIG. 21 shows bars are colored according to their tissue of origin—blue for synovial CD4 T cells, salmon for total blood CD4 T cells, red for naive blood CD4 T cells, and dark red for non-naive blood CD4 T cells. (A) Number of high-quality barcodes (each representing a cell) obtained for each sample. (B) Number of CD4 T cell clonotypes obtained for each sample. (C) Mean clone size (in number of cells) in each sample. (D) Median clone proportion (as a fraction of the sample total number of cells) for each sample.

FIG. 22 shows example of doublet detection for a single sample. The computed best distribution of the proportion of artificial nearest neighbors (pANN) for each of three choices of doublet generation strategy (whether additive or average, and whether normalized or not) are shown in the lower row, with the actual cells' pANN values in faded blue and the synthetic doublets' pANN values in faded red. It is intuitively clear that the best separation is achieved with additive doublet generation without normalization, and the AUC of the ROC in the upper row plots verifies this. Further supporting the accuracy of this doublet detection, overlaid in solid colors are the pANN distributions of subsets of the observed cells colored according to their number of TCRα and TCRβ chains. Cells with double-TCRα/double-TCRβ chains are colored red, and as expected given the biological improbability of such a state in a single cell (thus very high likelihood of being a doublet) these are far to the right of the pANN distribution, and thus excluded. Double-TCRα/single-TCRβ cells are more biologically plausible, and a greater portion of those (solid blue bars) are retained due to having low pANN, versus those that fall on the high-pANN side of the distribution. Essentially all of the single-TCRα/zero-TCRβ and zero-TCRα/single-TCRβ cells are classified as singlets.

FIG. 23 shows OLS for the relationship between CD4 repertoire mean CDR3 risk and RA HLA risk, computed for all measured samples (top left) and separately for subsets of samples based on their location of origin (blood vs synovium) or identity (naive vs non-naive in blood).

FIG. 24 shows the mean CDR3 risk score (±standard error of the mean) for clonotypes separated by their TRAJ, TRAV, TRBJ, and TRBV rearrangements as labeled, including clonotypes from all samples. The red line is the overall mean across all rearrangements.

FIG. 25 shows heatmaps of the predominant amino acid (letters superimposed on plot) at each position (horizontal axis) within each TRBJ rearrangement-defined subset of cells (vertical axis), for CDR3 sequences between length 12 and 18. A darker square reflects near-complete dominance at that position of the labeled amino acid, while a lighter square reflects greater diversity at that position. The sequences adjoining each heatmap are the amino acids comprising the proximal end of the corresponding row's TRBJ gene. Outlined boxes indicate CDR3 positions where the predominant amino acid was found to be associated with RA predisposing HLA-DRB1 alleles, with green indicating a negative association (less risky) and red indicating a positive association (more risky).

FIGS. 26 and 27 show heatmaps of the mean CDR3 risk score stratified by individual samples (vertical axis) and TRAJ, TRAV, TRBJ, or TRBV gene rearrangement (horizontal axis).

FIG. 28 show MANOVA test p-values for the multivariate multiple linear regression model (MMLM) analysis to detect associations between TRBJ usage and all alleles at an HLA site, to estimate the degree to which variation between individuals in TRBJ gene usage can be predicted by all alleles at an HLA site.

FIGS. 29A-E demonstrates HLA-DRB1 variation influences T cell repertoire TRBV and TRBJ gene usage. (FIG. 29A) Healthy individuals' bulk TCR repertoire and HLA genotypes were analyzed (data from [16]; n=628, ~1×105 T cells per subject). (FIG. 29B-FIG. 29C) Significance of association between T cell repertoire-wide (FIG. 29B) TRBV and (FIG. 29C) TRBJ usage and each of 369 HLA amino acid sites. (FIG. 29D) Explained variance in TCR repertoire frequency of each TRBJ gene (y-axis) by variation at each variable HLA-DRB1 site (x-axis). (FIG. 29E) TRBJ2-3 and TRBJ2-5 TCR repertoire frequency stratified by HLA-DRB1 allele dosage at select sites, demonstrating increased TRBJ2-3 frequency in individuals with leucine at HLA-DRB1 site 67.

FIGS. 30A-B demonstrates individuals with RA risk-associated HLA-DRB1 alleles have more T cells with TRB J2-3 and J2-5. (FIG. 30A) The frequency of circulating T cells containing specific germline-encoded TCR B subunits in each individual plotted against their HLA-DRB1 RA-associated risk score (n=628, ~1×105 T cells per subject). Significant associations with replication in an independent cohort shown with ordinary least square (OLS) fit with p-value indicated. (FIG. 30B) Similar analyses were performed for associations of TRBV and TRBJ gene usage with HLA-derived risk scores for two additional autoimmune conditions (CD, celiac disease, blue; TID, type 1 diabetes, green). For each significant association (corrected p ≤0.01), the magnitudes (B) of the significant V gene (circles) and J gene (star) associations with the corresponding HLA-based disease risk are plotted on the y axis, with the proportion of the full TCR repertoire containing the V or J gene rearrangement on the x axis. Across these three disorders, the only HLA-selected TRBJ genes are found in RA.

FIGS. 31A-G demonstrates germline-encoded TCR sequences influence HLA-DRB1 selection for CDR3 features (FIG. 31A) The TCR CDR3 amino acid motif preferentially selected by RA risk-associated HLA-DRB1 alleles (referred to here as the CDR3DRB1* motif [8]). Individual TCR sequences can be scored for similarity to the CDR3DRB1* motif. (FIG. 31B) The mean CDR3DRB1* motif scores for each TRBJ gene-defined CD4 T cell subset from 17 RA patients. (FIG. 31C) The CDR3 region (of length 13) for T cells with different TRB J genes are shown with the most common amino acid at each position labeled and the frequency of that amino acid indicated by the shade of blue. Amino acids outlined in red represent those found in the CDR3DRB1* motif, where as those in green are amino acids that were negatively associated with HLA-DRB1* selection. (FIG. 31D) A panel of 17 seropositive RA patients contributed peripheral blood from which CD4 T cells were isolated for TCR repertoire and HLA genotype data. (FIG. 31E) The relationship between the mean CDR3DRB1* score and the HLA-DRB1 RA risk for each of the 26 samples across the 17 RA subjects, showing the overall repertoire-wide HLA-DRB1* selection of CDR3 (p=0.0006). (FIG. 31F) For each TRBJ gene subset, the significance of the association between CDR3DRB1* score and HLA-DRB1-score shows the uniquely significant association in the TRBJ2-3 subset. (FIG. 31G) Comparison of the HLA/CDR3 relationship in the TRBJ2-3 subset (left) and the TRBV19 subset (right).

FIGS. 32A-D shows RA synovium demonstrates an enrichment of CD4 T cells with TRBJ2-3 TCRs relative to blood in RA patients. For each TRBJ gene, (FIG. 32A) shows the frequency of CD4 T cells with the TRBJ gene in each of naive blood (light red), non-naive blood (dark red), and synovium (blue) across all samples for which we have both blood and synovial repertoire data. Uniquely among TRBJ genes, TRBJ2-3 CD4 T cells are significantly more frequently found in synovium than in blood. (FIG. 32B) This effect is also shown in a GLMM analysis of TRBJ gene frequency in synovium versus blood conditioned on subject, with the effect significance shown in (FIG. 32C) and magnitude shown in (FIG. 32D), again demonstrating that synovium favors TRBJ2-3.

### DETAILED DESCRIPTION OF THE INVENTION

Provided herein are methods of using the T cell receptors (TCR) of CD4+ T cells (i.e., targetable subset of T cells), which feature is related to an individual's genetics with respect to their risk for Rheumatoid Arthritis (RA). The targetable subset of T cells comprises a TCR comprising T Cell Receptor Beta Joining 2-3 (TRBJ2-3). See also, ncbi.nlm.nih.gov/gene?Db=gene&Cmd=DetailsSearch&Term=28626; proteinatlas.org/ENSG00000211767-TRBJ2-3; and proteinatlas.org/ENSG00000211767-TRBJ2-3/single+cell, which are incorporated herein by reference.

Here, we first find that HLA variation associates with the overall frequencies of germline-encoded TRB V and J genes in the TCR repertoire in healthy subjects, with the most significant effect at HLA-DRB1 positions that associate with RA risk. Again in healthy subjects, we then tested for associations between HLA variation and the frequency of each individual TRBV or J gene, finding that the HLA effect on repertoire TRBJ usage is also concentrated in HLA-DRB1 positions that associate with RA risk, and that the most-influenced TRBJ genes are TRBJ2-3 and TRBJ2-5. Third, using a composite measure of RA-related HLA-DRB1 variation, we find that among healthy subjects a higher burden of RA-associated HLA-DRB1 alleles increases the TCR repertoire frequencies of TRBJ2-3 and TRBJ2-5. The TRBJ2-3 CD4 T cell subset overlaps with a CDR3 feature independently selected by HLA-DRB1, which is unique across all TRBV and TRBJ-defined subsets. Finally, with TCRseq data obtained from a panel of RA patients, we show that the HLA-DRB1 selection of TCR CDR3 features is concentrated in the TRBJ2-3 subset of the TCR repertoire, and that CD4 T cells with the TRBJ2-3 sequence are preferentially present at the site of disease activity in the synovium. Taken together, our results suggest the TCR interaction with the antigen-HLA complex is in part shaped by interactions between the germline-encoded segments of the TCR, and that generate broad patterns in an individual TCR repertoires, including enriched TRBJ2-3 encoded T cells in RA patients with high risk HLA-DRB1 alleles.

Provided herein is a method of treatment of Rheumatoid Arthritis (RA), the method comprising administering to the subject a therapeutically effective amount of a compound which targets and/or depletes CD4+ T cells comprising T cell receptor (TCR) that binds to HLA-DRB1 antigen. In certain embodiments, the method comprises administering a compound which targets and/or depletes CD4+ T cells comprising T cell receptor beta joining 2-3 (TRBJ2-3). In certain embodiments, the method comprises administering a compound which targets and/or depletes CD4+ T cells comprising T cell receptor beta variable 19 (TRBV19). In certain embodiments, the method comprises administering a compound which targets and/or depletes CD4+ T cells comprising T cell receptor beta variable 20-1 (TRBV20-1). In certain embodiments, the method comprises administering a compound which targets and/or depletes CD4+ T cells comprising T cell receptor beta variable 15-1 (TRBV2-1).

In certain embodiments, provided herein is a method of treating RA, the method comprising administering a therapeutically effective amount of an immunoglobulin, wherein the immunoglobulin specifically targets and/or binds TRBJ2-3 of the CD4+ T cells. In certain embodiments, the immunoglobulin targets HLA-DRB1 antigen binding region of the CD4+ T cells. In certain embodiments, the immunoglobulin targets C-terminal aspartic acid (D) amino acid of a complementarity-determining region 3 (CDR3) of the T cell receptor of the T cell. In certain embodiments, the immunoglobulin depletes CD+T cells in a dose-dependent manner.

In certain embodiments, the compound is an antisense oligonucleotide (ASO), RNAi, small interfering RNA (siRNA), or microRNA (miRNA).

In certain embodiments, the compound is administered systemically. In certain embodiments, the compound is administered intravenously. In certain embodiments, the compound is administered via a synovial injection. In certain embodiments, the compound is administered to a subject diagnosed with RA. In certain embodiments, the compound is administered to a subject at risk for RA (e.g., genetically predisposed to RA).

In certain embodiments, the method comprises administering an immunoglobulin to a subject diagnosed with RA or at risk of RA, and monitoring of T cell counts and RA disease activity. The results would include measures of RA disease activity such as swollen and tender joints, systemic markers of inflammation, and damage to the joints (as may be measured on X-ray or ultrasound imaging); as well as monitoring for safety of the treatment for instance with rates of infection.

Without wishing to be bound by theory, targeting a subset of T cells (e.g., comprising TCR comprising TRBJ2-3) provides greater RA treatment efficacy by focusing the effect in an area more likely to be underlying the disease in specific patients, and greater safety by having a more tailored effect minimizing overall immune suppression.

The term “immunoglobulin” is used herein to include antibodies, functional fragments thereof, and immunoadhesins. Antibodies may exist in a variety of forms including, for example, polyclonal antibodies, monoclonal antibodies, camelized single domain antibodies, intracellular antibodies (“intrabodies”), recombinant antibodies, multi-specific antibody, antibody fragments, such as, Fv, Fab, F(ab)2, F(ab)3, Fab′, Fab′-SH, F(ab′)2, single chain variable fragment antibodies (scFv), tandem/bis-scFv, Fc, pFc′, scFvFc (or scFv-Fc), disulfide Fv (dsfv), bispecific antibodies (bc-scFv) such as BiTE antibodies; camelid antibodies, resurfaced antibodies, humanized antibodies, fully human antibodies, single-domain antibody (sdAb, also known as NANOBODY®), chimeric antibodies, chimeric antibodies comprising at least one human constant region, and the like. “Antibody fragment” refers to at least a portion of the variable region of the immunoglobulin that binds to its target, e.g., the cell receptor.

The “effective amount” of the compound is an amount sufficient to reduce or eliminate CD4 positive T cells comprising TRBJ2-3, TRBV19, TRBV20-1 and/or TRBV2-1. In certain embodiments, for a protein or peptide antagonist, e.g., antibody, antibody fragment or recombinant protein or peptide, the effective amount can be about 0.01 to 25 mg antibody/injection. In one embodiment, the effective amount is 0.01 to 10 mg antibody/injection. In another embodiment, the effective amount is 0.01 to 1 mg antibody/injection. In another embodiment, the effective amount is 0.01 to 0.10 mg antibody/injection. In another embodiment, the effective amount is 0.2, 0.5, 0.8, 1.0, 1.2, 1.4, 1.6, 1.8, 2.0, 2.2, 2.4, 2.6, 2.8, 3.0 up to more than mg antibody/injection. Still other doses falling within these ranges are expected to be useful. In one embodiment an effective amount for the nucleic acid and/or protein inhibitor of composition (a) includes without limitation about 0.001 to about 25 mg/kg subject body weight. In one embodiment, the range of effective amount is 0.001 to 0.01 mg/kg body weight. In another embodiment, the range of effective amount is 0.001 to 0.1 mg/kg body weight. In another embodiment, the range of effective amount is 0.001 to 1 mg/kg body weight. In another embodiment, the range of effective amount is 0.001 to 10 mg/kg body weight. In another embodiment, the range of effective amount is 0.001 to 20 mg/kg body weight. In another embodiment, the range of effective amount is 0.01 to 25 mg/kg body weight. In another embodiment, the range of effective amount is 0.01 to 0.1 mg/kg body weight. In another embodiment, the range of effective amount is 0.01 to 1 mg/kg body weight. In another embodiment, the range of effective amount is 0.01 to 10 mg/kg body weight. In another embodiment, the range of effective amount is 0.01 to 20 mg/kg body weight. In another embodiment, the range of effective amount is 0.1 to 25 mg/kg body weight. In another embodiment, the range of effective amount is 0.1 to 1 mg/kg body weight. In another embodiment, the range of effective amount is 0.1 to 10 mg/kg body weight. In another embodiment, the range of effective amount is 0.1 to 20 mg/kg body weight. In another embodiment, the range of effective amount is 1 to 25 mg/kg body weight. In another embodiment, the range of effective amount is 1 to 5 mg/kg body weight. In another embodiment, the range of effective amount is 1 to 10 mg/kg body weight. In another embodiment, the range of effective amount is 1 to 20 mg/kg body weight. Still other doses falling within these ranges are expected to be useful.

In certain embodiments, an effective amount may be determined based on an animal model, rather than a human patient.

As used herein, the term “a therapeutically effective amount” refers an amount sufficient to achieve the intended purpose. For example, an effective amount of a therapy for rheumatoid arthritis (RA) is sufficient to target and/or deplete CD4 positive (+) T cells comprising T cell receptor beta joining 2-3 (TRBJ2-3), T cell receptor beta variable 19 (TRBV19), T cell receptor beta variable 20-1 (TRBV20-1), or T cell receptor beta variable 15-1 (TRBV2-1) in a subject. An effective amount for treating or ameliorating a disorder, disease, or medical condition is an amount sufficient to result in a reduction or complete removal of the symptoms of the disorder, disease, or medical condition. The effective amount of a given therapeutic agent will vary with factors such as the nature of the agent, the route of administration, the size and species of the animal to receive the therapeutic agent, and the purpose of the administration. The effective amount in each individual case may be determined by a skilled artisan according to established methods in the art.

As used herein, “disease”, “disorder” and “condition” are used interchangeably, to indicate an abnormal state in a subject.

As used herein, the terms “Patient” or “subject” or “individual” means a mammalian animal, including a human, a veterinary or farm animal, a domestic animal or pet, and animals normally used for clinical research. In one embodiment, the subject of these methods and compositions is a human. In one embodiment, the subject has RA. In another embodiment, the subject has an early stage of RA and has yet to be treated with any therapy. In another embodiment, the subject has RA and is being treated with conventional methodologies, e.g., administration of anti-inflammatories, but is not responding to the treatment optimally or in a manner sufficient to achieve a sufficient therapeutic benefit. In another embodiment, the subject has advanced RA beyond the early stages.

As used herein, the term “administration” or any grammatical variations thereof refers to delivery of composition described herein to a subject. By “administration” or “routes of administration” include any known route of administration that is suitable to the selected inhibitor or composition, and that can deliver an effective amount to the subject. In one embodiment of the methods described herein, the routes of administration include one or more of oral, parenteral, intravenous, intra-nasal, sublingual, by inhalation or by injection directly into the site of the RA.

As used throughout this specification and the claims, the terms “comprise” and “contain” and its variants including, “comprises”, “comprising”, “contains” and “containing”, among other variants, is inclusive of other components, elements, integers, steps and the like. The term “consists of” or “consisting of” are exclusive of other components, elements, integers, steps and the like.

It is to be noted that the term “a” or “an”, refers to one or more, for example, “an enhancer”, is understood to represent one or more enhancer(s). As such, the terms “a” (or “an”), “one or more,” and “at least one” is used interchangeably herein.

With regard to the description of various embodiments herein, it is intended that each of the compositions herein described, is useful, in another embodiment, in the methods of the invention. In addition, it is also intended that each of the compositions herein described as useful in the methods, is, in another embodiment, itself an embodiment of the invention.

Unless defined otherwise in this specification, technical and scientific terms used herein have the same meaning as commonly understood by one of ordinary skill in the art to which this invention belongs and by reference to published texts, which provide one skilled in the art with a general guide to many of the terms used in the present application.

### Specific Embodiments:

1. A method for treating rheumatoid arthritis (RA), the method comprising administering to the subject a therapeutically effective amount of a compound that targets and/or depletes CD4 positive (+) T cells comprising T cell receptor beta joining 2-3 (TRBJ2-3), T cell receptor beta variable 19 (TRBV19), T cell receptor beta variable 20-1 (TRBV20-1), or T cell receptor beta variable 15-1 (TRBV2-1).

2. The method of embodiment 1, wherein the compound depletes CD4+ T cells comprising TRBJ2-3.

3. The method of embodiment 1 or embodiment 2, wherein the compound is an immunoglobulin, optionally an antibody or an antibody fragment, optionally a monoclonal antibody, optionally a humanized monoclonal antibody.

4. The method of any one of embodiments 1 to 3, wherein the compound is an immunoglobulin that targets to TRBJ2-3.

5. The method of any one of embodiments 1 to 3, wherein the compound is an immunoglobulin that targets C-terminal aspartic acid (D) amino acid of a complementarity-determining region 3 (CDR3) of the T cell receptor of the T cell.

6. The method of any one of embodiments 1 to 5, wherein the compound is administered systemically, optionally intravenously.

7. The method of any one of embodiments 1 to 5, wherein the compound is administered via a synovial injection.

8 The method of embodiment 1 or embodiment 2, wherein the compound is an antisense oligonucleotide (ASO), RNAi, small interfering RNA (siRNA), or microRNA (miRNA).

### EXAMPLES

The following examples are provided to illustrate certain aspects of the claimed invention. The invention is not limited to these examples.

### Example 1

### HLA-DRB1 Rheumatoid Arthritis (RA) Risk Alleles Preferentially Select TRBJ2-3-containing CD4 T Cells in RA Patients

Purpose: The largest genetic risk factor for RA localizes to the MHC Class II HLA-DRB1 gene, which encodes the machinery for antigen presentation to CD4 T cells. The mechanisms by which CD4 T cells may be involved in this elevated risk is unclear. To elucidate potential pathogenic mechanisms, here we further explored the recent demonstration by Ishigaki et al. (2022) that in healthy individuals, the presence of high-risk RA-associated HLA-DRB1 alleles results in thymic selection of specific patterns of T cell receptors (TCRs) in the CD4 T cell repertoire, and that those patterns are enriched among CD4 T cell clones that recognize citrullinated antigens.

Methods: We obtained synovial tissue (n=15) and blood (n=17) from patients (n=19), among which 13 were paired, satisfying either the 1987 or 2010 ACR/EULAR RA classification criteria, with informed consent and IRB approval. We isolated CD4 T cells and performed single-cell transcriptome and TCR sequencing (10X platform) with yield of ~5,000-9,000 cells per sample. We applied a custom computational pipeline to isolate high-quality single-cell data, infer HLA-DRB1 genotypes, and quantify TCR repertoire metrics including clonal expansion and the Ishigaki et al. TCRβ CDR3 risk score. With this dataset and public CD4 TCR and HLA data of healthy individuals' blood (Emerson et al. 2017), we also computed associations between V and J gene usage and HLA-DRB1 genotype.

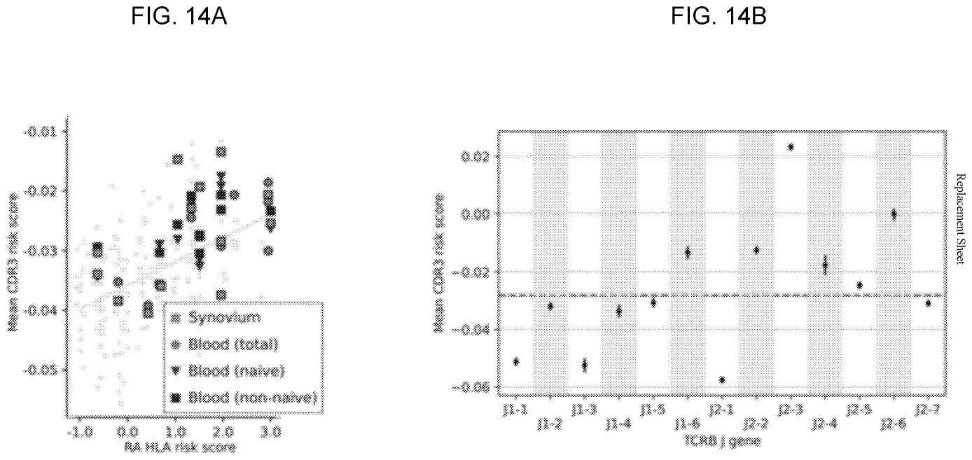

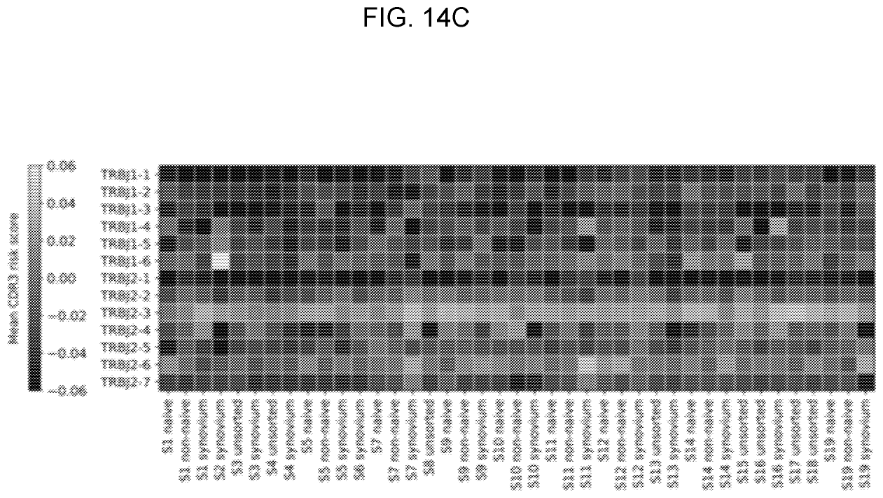

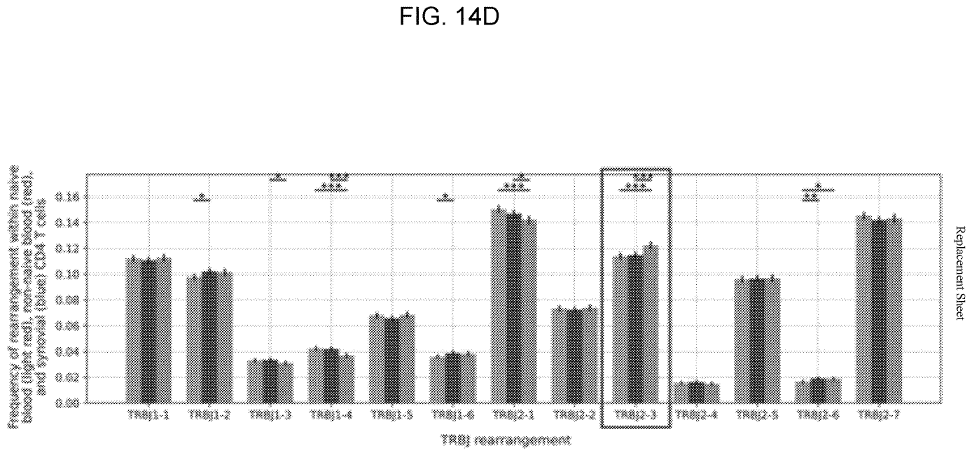

Results: Here we identified CD4 TCR clones harboring TCRβ CDR3 features selected by HLA-DRB1 risk alleles in RA patients, in increasing abundance with increasing risky HLA allele burden (FIG. 14A). In an effort to define additional features of clones with features selected by the high-risk HLA-DRB1 alleles, we noted a highly significant enrichment of clones containing the TRBJ2-3 gene rearrangement (FIG. 14B), which is consistent across all RA patients (FIG. 14C).

Examining the portion of the CDR3 regions overlapping with the germline-encoded TRBJ2-3 gene, we detected negatively charged TCR amino acids that are predicted to interact with the HLA-DRB1 risk-encoded amino acids. In the synovium of RA patients, we demonstrate that across nearly one hundred thousand CD4 T cells, the size of each CD4 T cell clone is small, below 1% of the total CD4 population. Nonetheless, in comparison to all other TRBJ genes, we detected enrichment of the TRBJ2-3 CD4 T cell clones in RA synovium (FIG. 14D).

FIG. 14(A) The relationship between mean CDR3 risk score (reflecting the presence of TCRβ CDR3 features selected by HLA-DRB1) and the burden of RA-associated HLA-DRB1 alleles (quantified as the RA HLA risk score) across 19 RA patients shows an increasing abundance of those TCR features with increasing risky HLA allele burden. FIG. 14(B) The subset of CD4 T cells with the TRBJ2_3 rearrangement has a markedly higher enrichment of clones with those features across all RA patients, and FIG. 14(C) this is observed in each RA patient individually as well. FIG. 14(D) The frequency of CD4 T cell clones with TRBJ2_3 is higher in RA synovium than blood, uniquely across all TRBJ rearrangements.

Conclusion: Our study has defined TCR receptor repertoire patterns that associate with high-risk HLA-DRB1 alleles in RA patients, and identified the CD4 T cell subset with TRBJ2-3 as containing a particularly high concentration of those patterns. Given the small clone sizes for CD4 T cells in general, and that TRBJ2-3-containing CD4 clones are enriched across the CD4 T cell repertoire in individuals with risky HLA-DRB1 alleles, we argue that CD4 T cell involvement in RA synovial pathology may be greatly influenced by germline-encoded features distributed across the CD4 T cell repertoire. These findings may motivate therapeutic targeting of CD4 T cell subsets in RA patients that are defined by their germline-encoded regions, including TRBJ2-3.

### Example 2

### Rheumatoid Arthritis-associated HLA-DRB1 Alleles Select TRBJ 2-3-containing CD4 T Cells

### Introduction

Rheumatoid arthritis (RA) is a systemic autoimmune disease, with a global prevalence estimated at 200-400/100,000 [1], in which the immune system is inappropriately activated by self tissues and causes damage and disability, principally centered on the synovium [2]. Although the development of RA is clearly multifactorial, it has a strong association with HLA-DRB1 [3]. Despite the unambiguous association of specific HLA-DRB1 alleles with RA [4] (which we denote collectively as HLA-DRB1*), the mechanisms by which variation in HLA-DRB1 influences disease risk-along with the implications of those mechanisms for understanding RA pathophysiology and suggesting therapeutic targets-remain unclear.

Chief, among its known functions, HLA-DRB1 is part of the MHC Class II complex that presents antigen to CD4 T cells. Each CD4 T cell has on its surface a heterodimeric T cell receptor (TCR, most often comprising one TCRα and one TCRβ chain) that recognizes specific antigen-HLA complexes. In order to recognize the wide spectrum of potential antigens, each individual's CD4 population has a highly diverse TCR repertoire generated by recombination of germline-encoded sequences (VJ recombination for TCRα, VDJ recombination for TCRβ), further diversified by nucleotide insertion and deletion at a region known as CDR3 complementarity-determining region 3) that is located at the VJ junction in TCRα and across the VDJ junction in TCRβ. A single CD4 T cell clonotype is thus defined by specific germline-encoded sequences (for the TCRβ chain, one each of approximately 45 TRBV genes, 2 TRBD genes, and 13 TRBJ genes) and the sequence of the CDR3 region (defined conventionally as the segment between the distal cysteine of the V gene and the proximal phenylalanine of the J gene). Theoretical estimates of TCR diversity are on the order of 10¹⁵ possible clonotypes [5], which exceeds the approximately 10¹² T cells in a typical human [6]. Candidate TCR are evaluated in the thymus by positive selection (for TCR capable of transducing signal) and negative selection (against TCR that recognize self antigen too strongly), and only the subset of candidate CD4 T cells with TCR passing those selection criteria are allowed to exit the thymus and enter the circulation as naive CD4 T cells.

The antigen presentation role of HLA-DRB1 is both peripheral-in the circulating blood and organs, where the HLA-DRB1 genotype of an individual plays a role in promoting recognition of specific antigens—and central—in the thymus, where the HLA-DRB1 genotype selects specific patterns in the TCR repertoire, shaping which potential antigen (and auto-antigen) specificities will ultimately be present in the circulating T cell population. We leverage our knowledge of the HLA-DRB1 alleles predisposing to RA to identify features of the TCR repertoire associated with HLA-DRB1* through the central mechanism, which could mark subsets of the CD4 T cell compartment as candidates for intermediating the disease process.

Specifically, in this report we first ask if there is a connection between disease-associated HLA-DRB1 alleles and the germline-encoded regions of the TCR repertoire. We then assess to what degree that connection may be related to previously-described selection of CDR3 features by HLA-DRB1. Finally, we evaluate in RA patients if the HLA-DRB1-selected germline-encoded TCR features are preferentially present at the site of disease activity in the synovium.

Our results suggest a model for the TCR interaction with the antigen-HLA complex that is shaped by interactions between HLA and the germline-encoded sections of TCR, and that points toward a feasible therapeutic targeting of a specific TRBJ gene in RA.

### Results

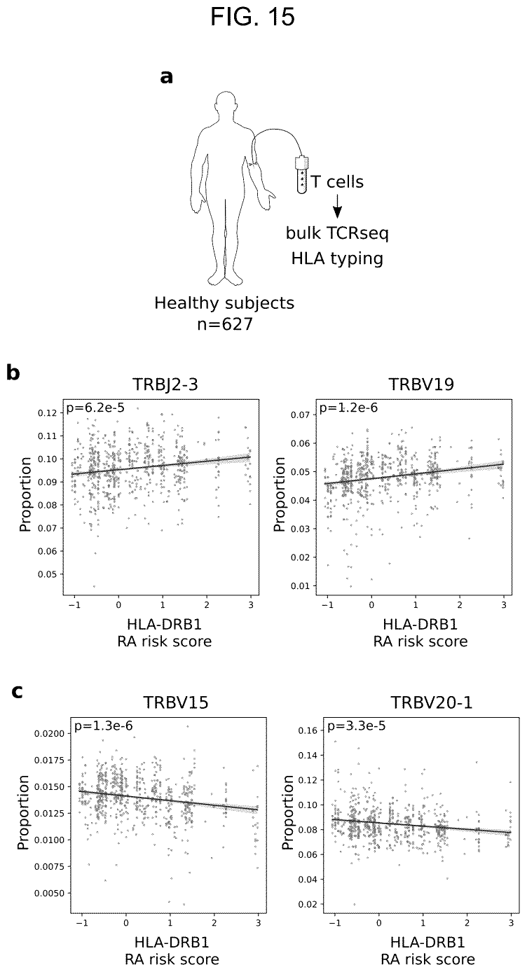

Previously [8], we analyzed bulk T cell repertoire and HLA genotype data of a large panel of healthy patients (without a known diagnosis of autoimmune disease) [7] to identify features of the TCR CDR3 region that were significantly either more or less frequently observed in the repertoires of individuals with HLA-DRB1 *. That analysis explicitly excluded the contribution of germline-encoded sequence to the CDR3 region (specifically, the overlap between the proximal CDR3 and the TRBV gene rearrangement, and between the distal CDR3 and the TRBJ gene rearrangement). Here we determine whether the TRBV or TRBJ gene rearrangements themselves are associated with HLA-DRB1* (FIG. 15A).

FIG. 15: Individuals with RA risk HLA-DRB1 alleles have more CD4 T cells with a J2-3 encoded TCR. (A) Bulk blood TCRseq data and HLA-DRB1 typing were obtained from a large previously characterized cohort of healthy subjects [7]. Plots in (B) and (C) show the proportion of the CD4 repertoire with the indicated V or J gene rearrangement versus the HLA-DRB1 RA risk score for each of the healthy subjects, with the OLS fit and p-value indicated. Significant associations indicate selection by HLA-DRB1 alleles.

A multivariate multiple linear regression model (MMLM) found the most significant association of TRBJ and TRBV usage with the HLA-DRB1 residues most strongly associated with RA, justifying the use of HLA-DRB1* in assessing these TRBV and TRBJ associations (FIG. 28).

FIG. 28: MANOVA test p-values for the multivariate multiple linear regression model (MMLM) analysis to detect associations between TRBJ usage and all alleles at an HLA site, to estimate the degree to which variation between individuals in TRBJ gene usage can be predicted by all alleles at an HLA site.

We discovered five TRBV or TRBJ gene associations with HLADRB1 significant at the level of p=0.01 (Bonferroni-corrected), of which four are shown in FIG. 15B-C. Of these, the only significantly-associated TRBJ rearrangement is TRBJ2-3, which is observed more frequently in subjects with HLA-DRB1*.

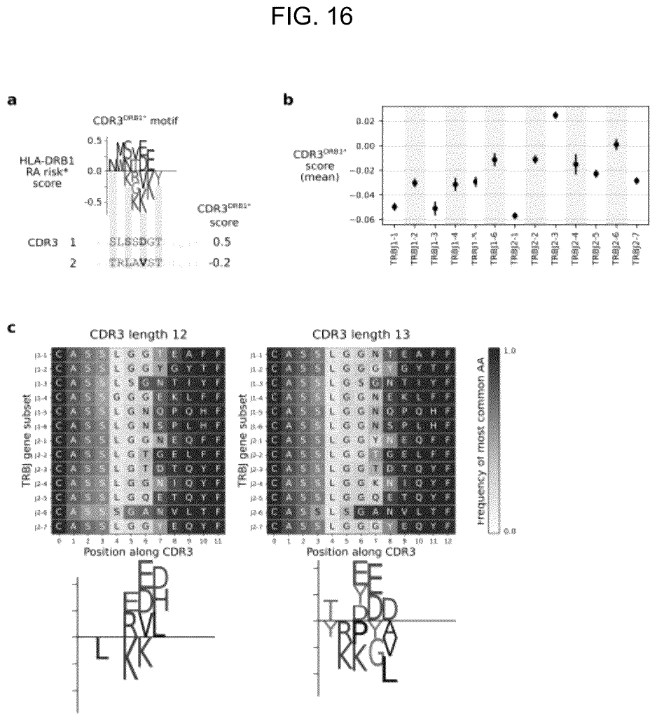

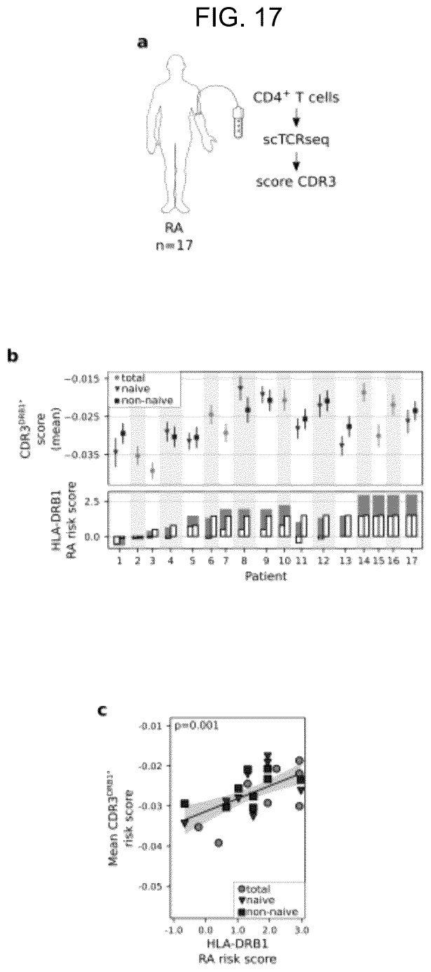

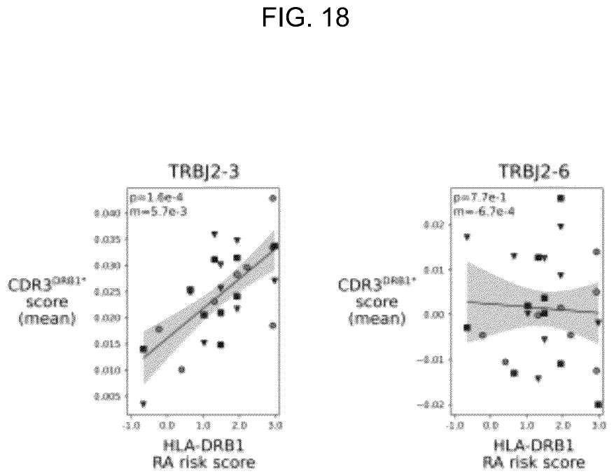

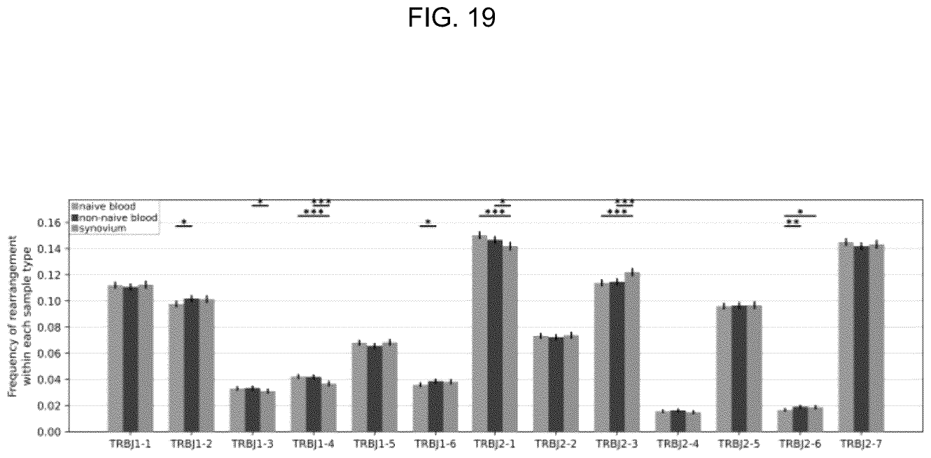

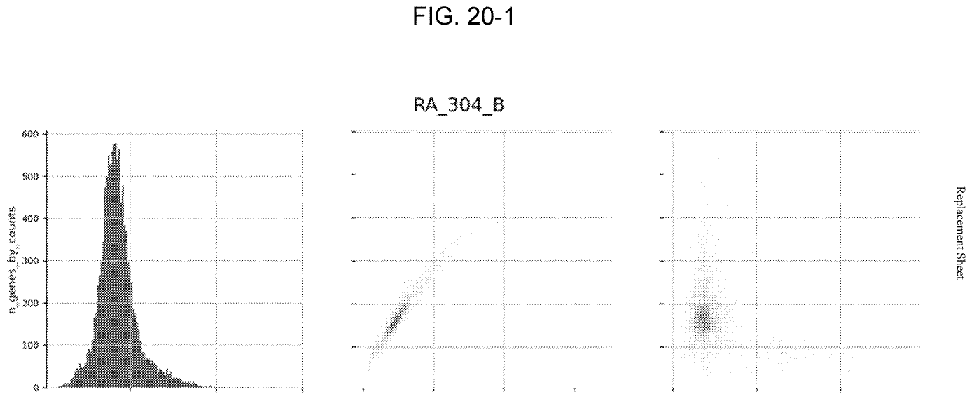

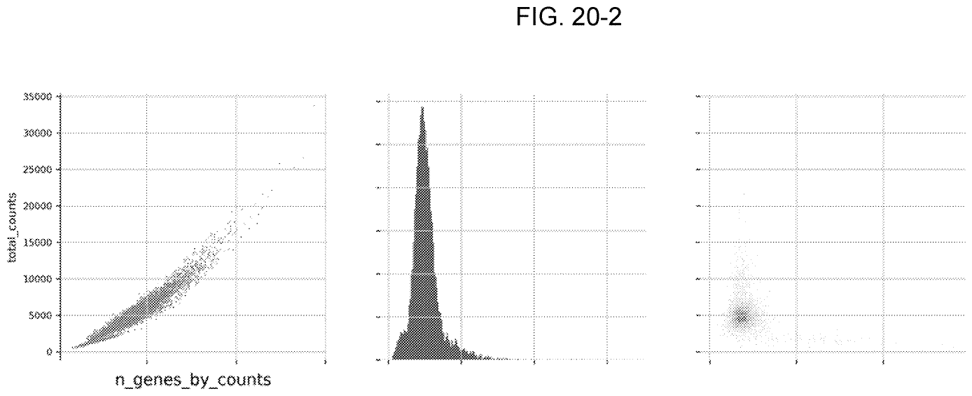

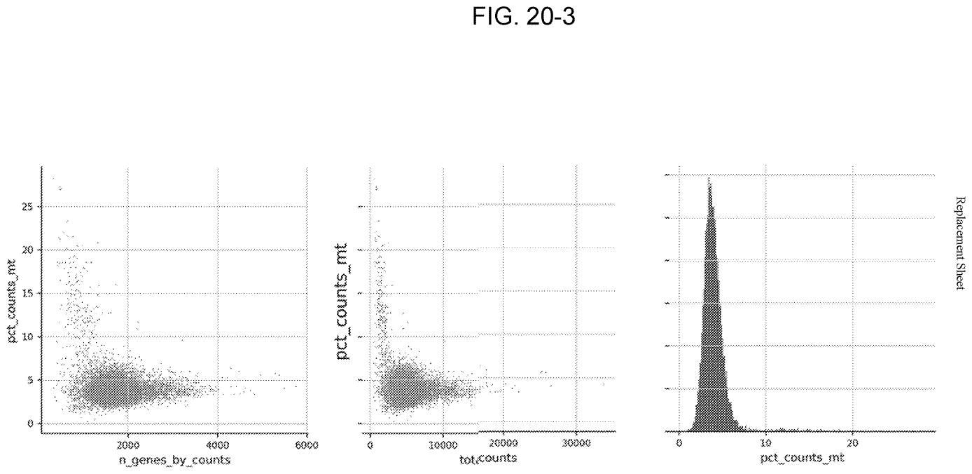

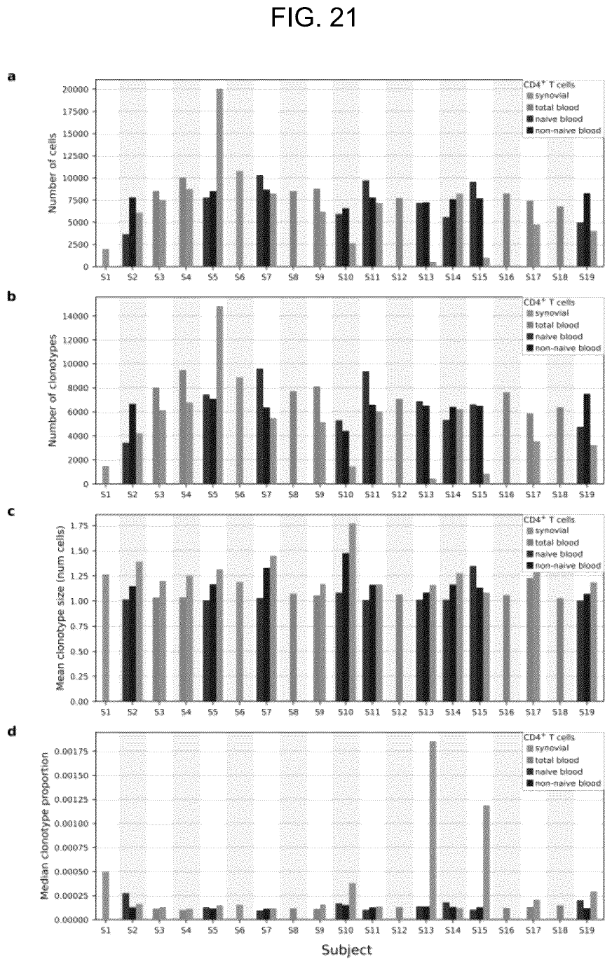

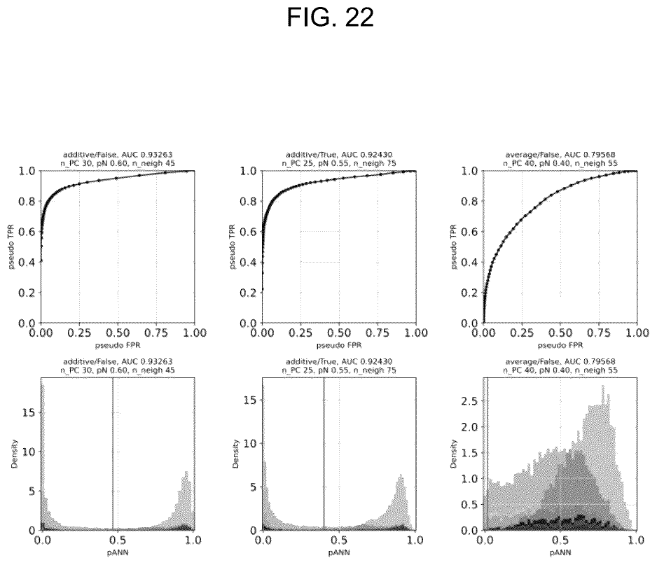

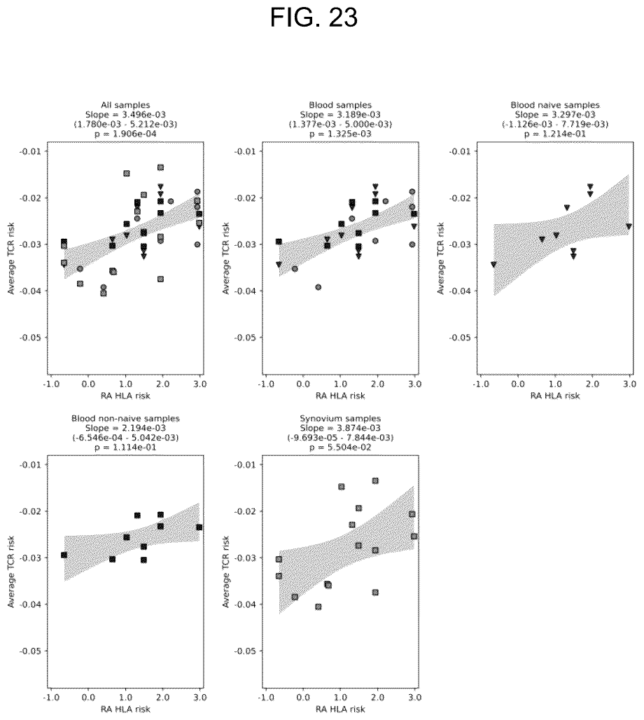

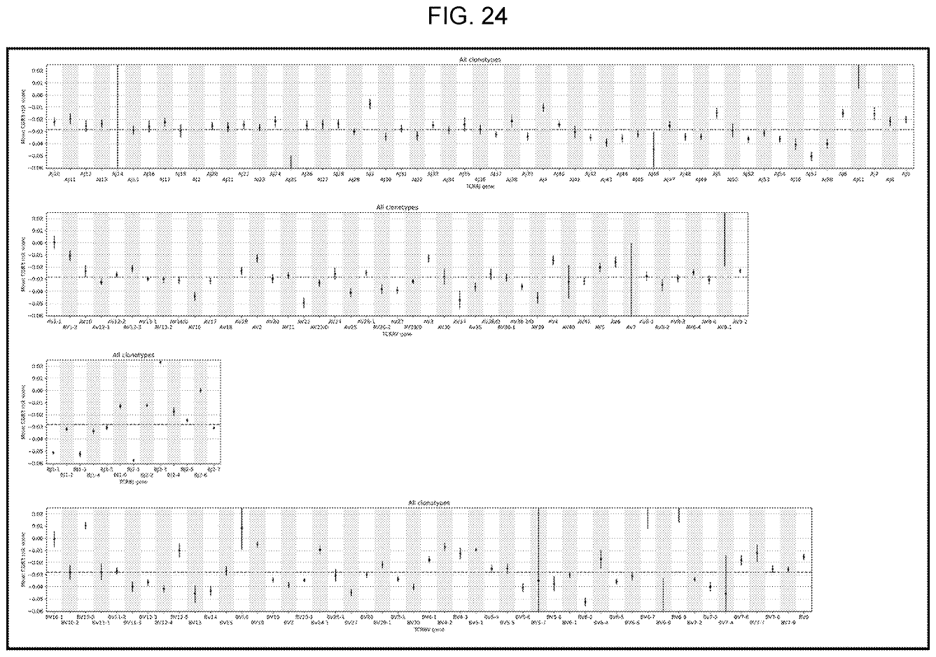

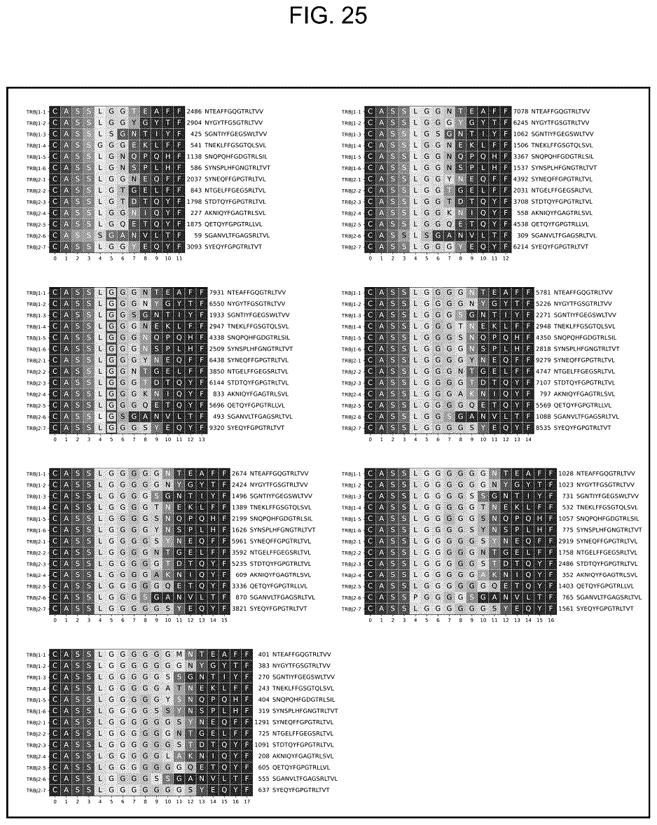

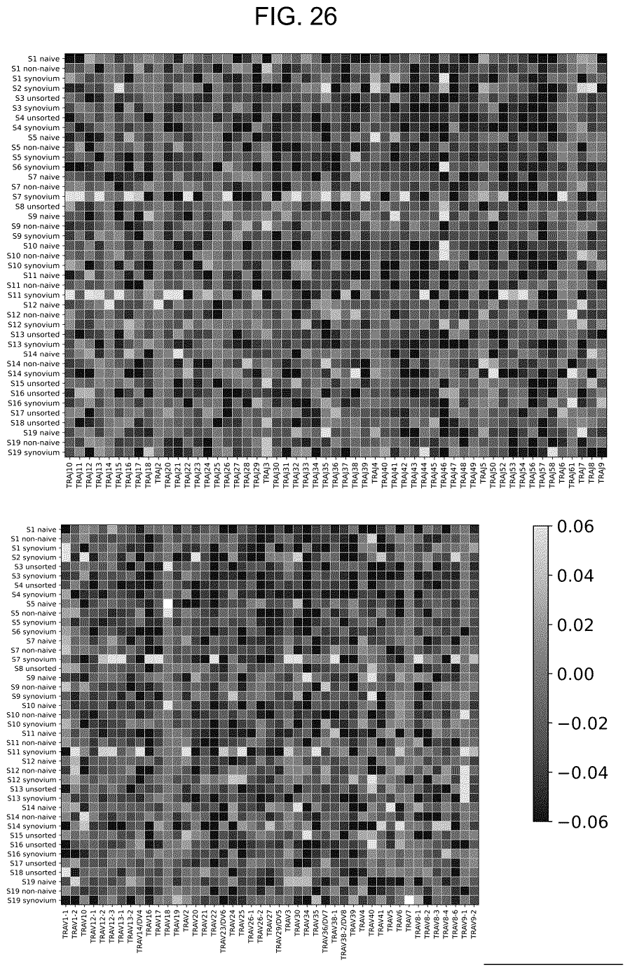

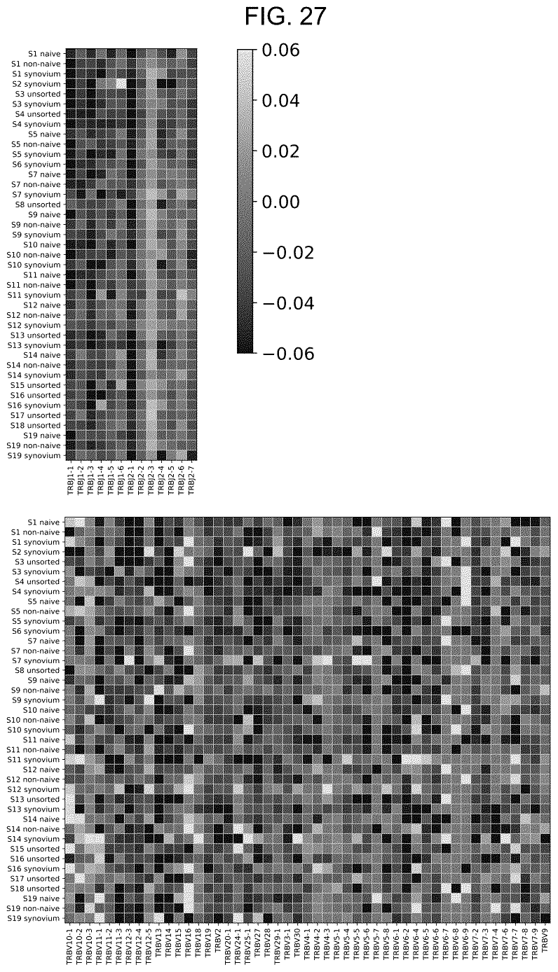

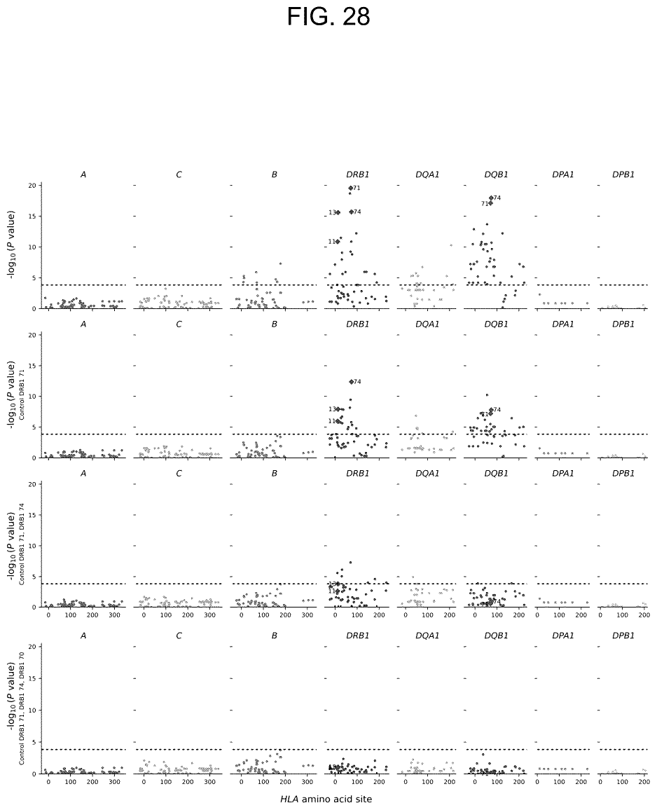

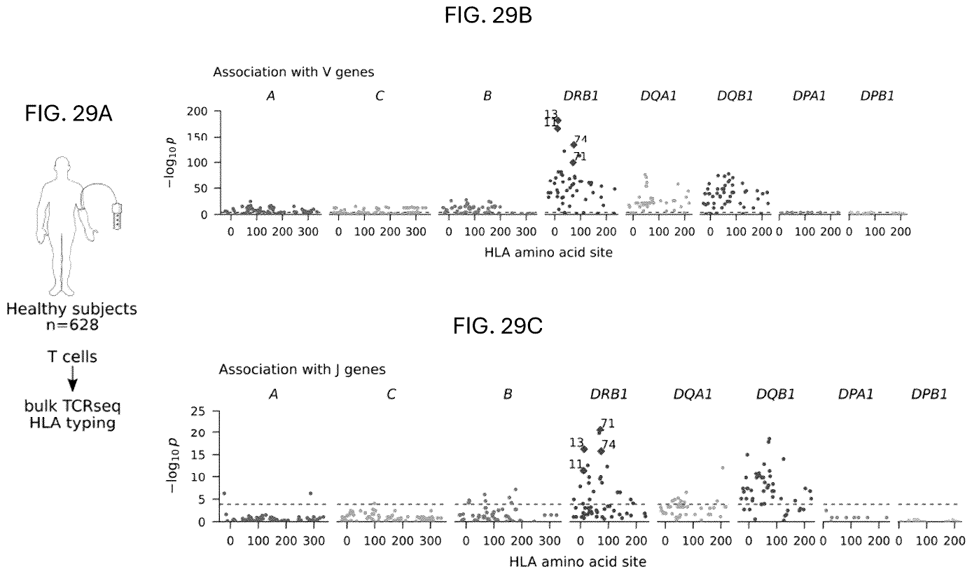

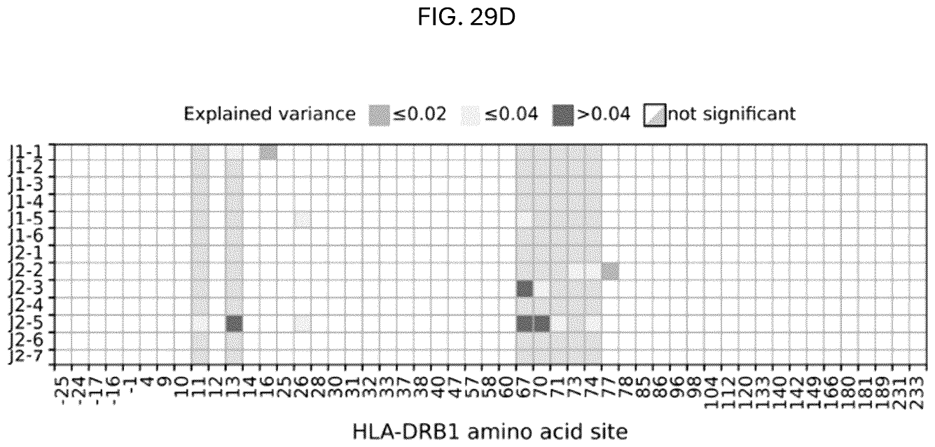

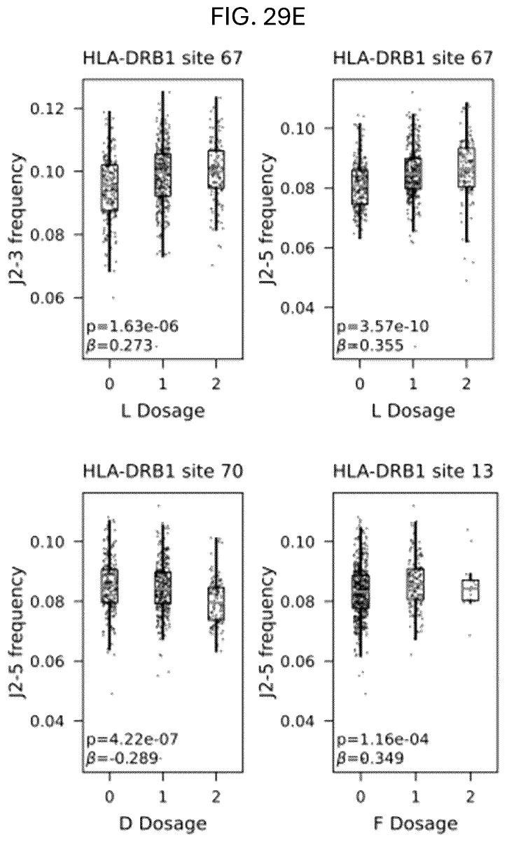

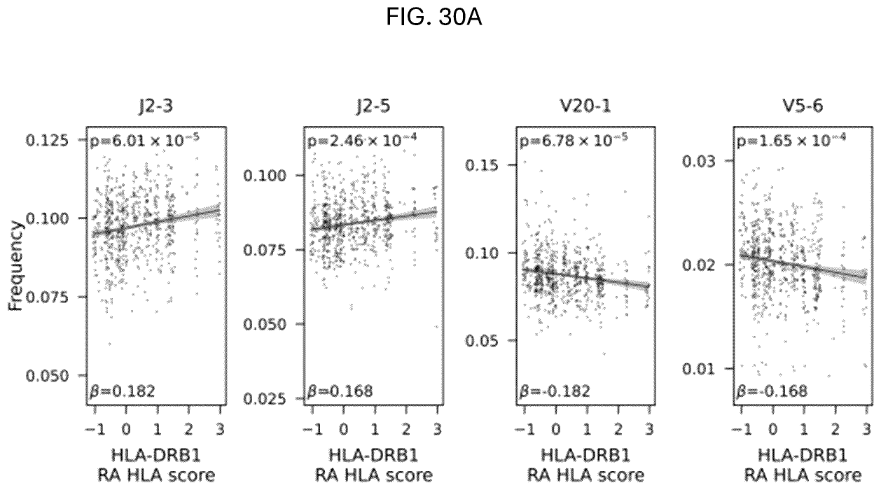

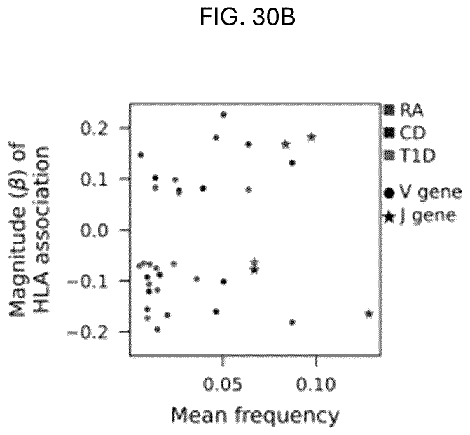

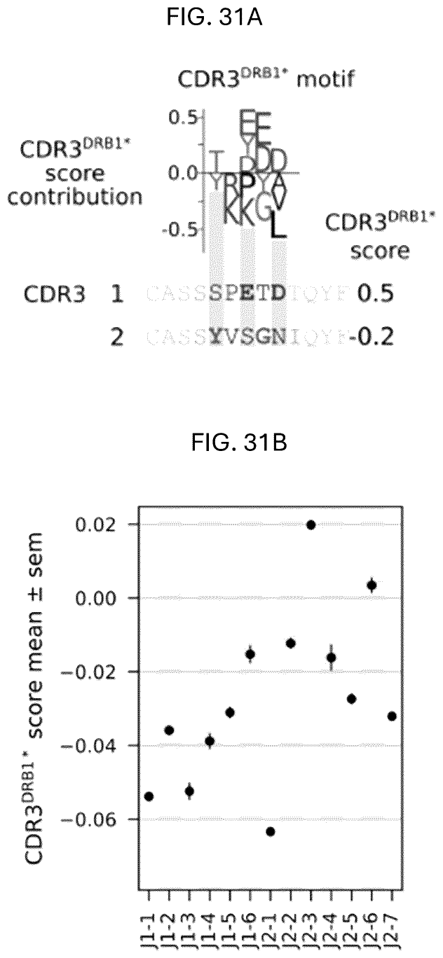

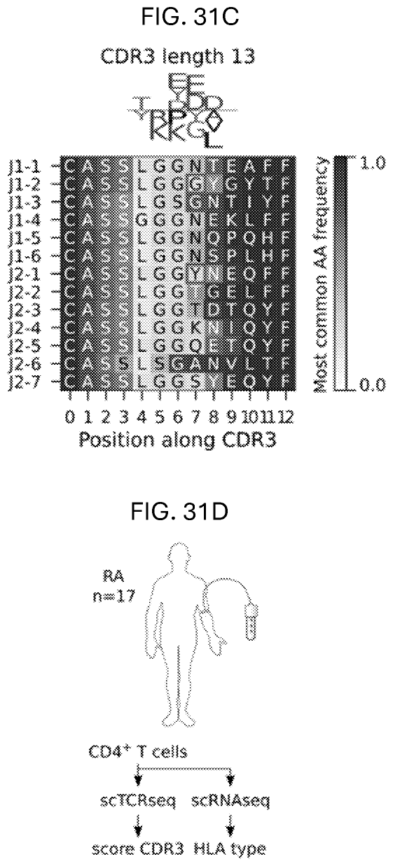

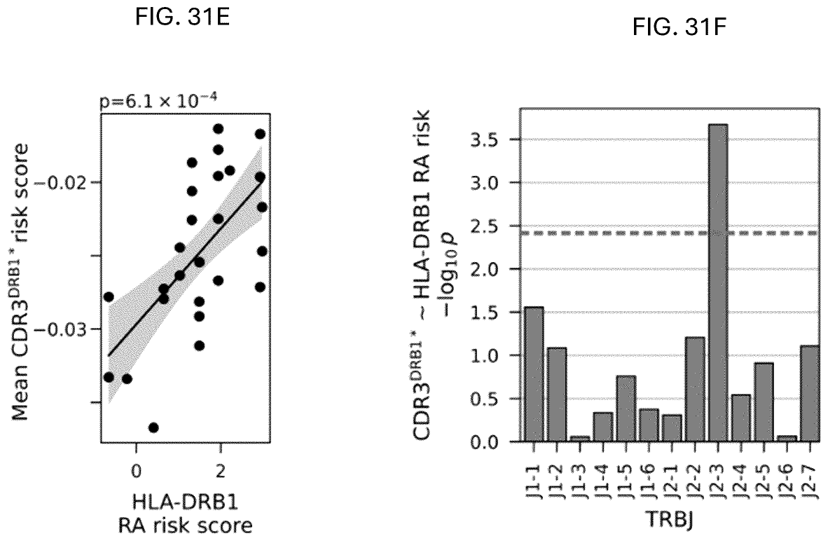

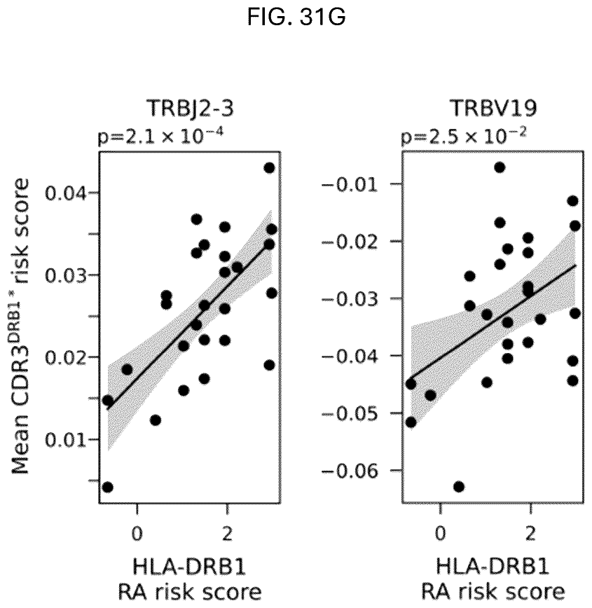

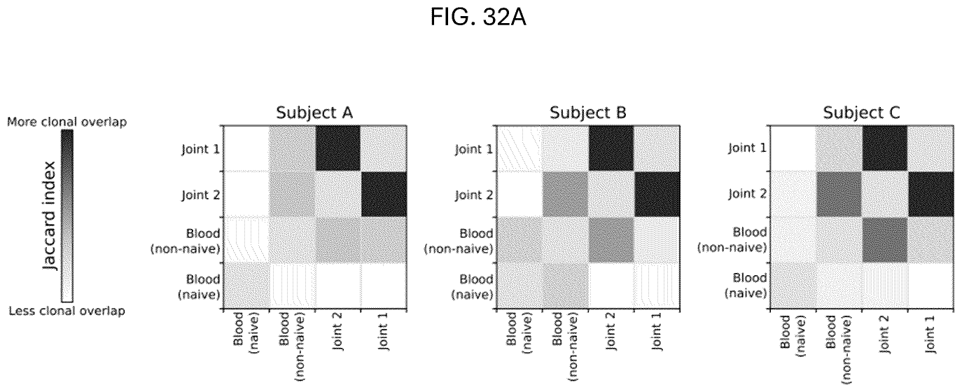

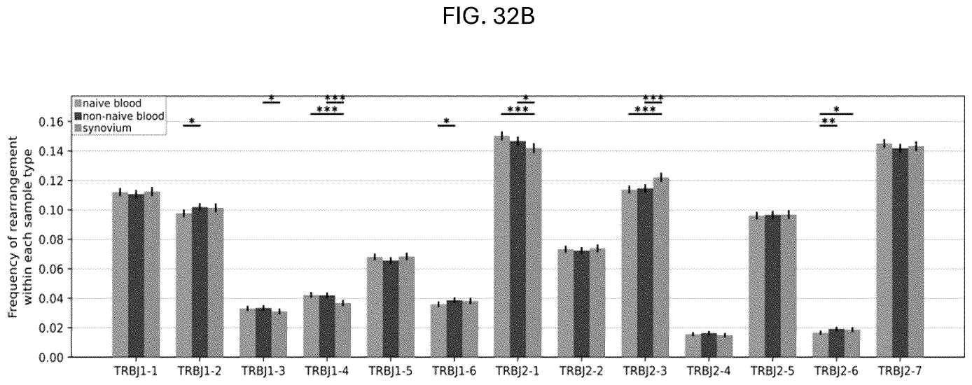

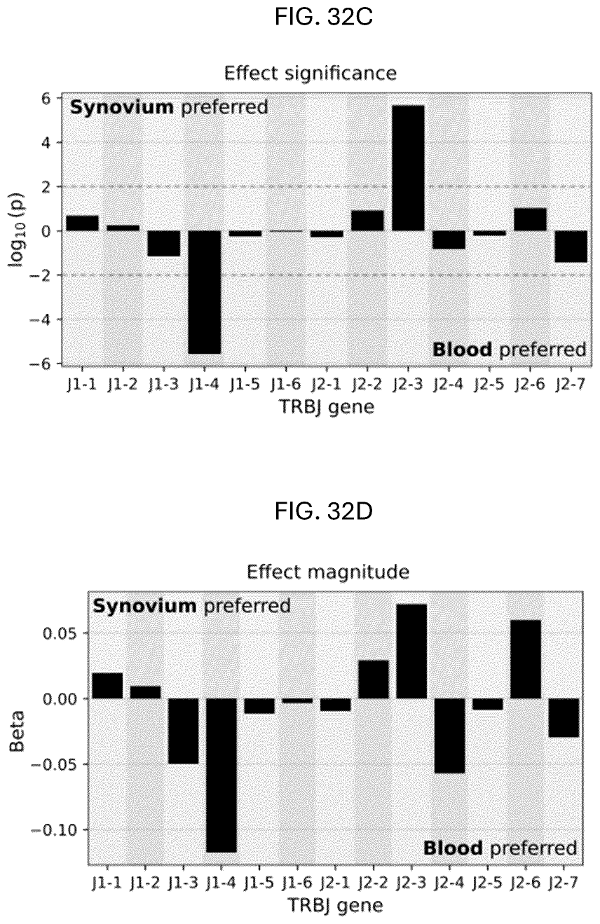

The CDR3 features selected by HLA-DRB1* can be identified and summed across an observed CDR3 sequence to yield a CDR3DRB1* score for the TCR, which reflects the overall degree to which a CD4 clonotype has HLA-DRB1*-selected features (FIG. 16A). Stratifying CD4 clones by their TRBJ rearrangements, we find that the subset of CD4 clones with the TRBJ2-3 rearrangement has markedly higher CDR3DRB1* scores than all others, suggesting that there is particularly strong HLA-DRB1* selection for those clones (FIG. 16B). The TRBJ2-6-containing clones had the next highest CDR3DRB1* selection score. We next examined a potential biochemical basis for why the HLA-DRB1* complexes preferentially select TRBJ2-3-containing CD4 clones. Noting that the distal CDR3 region overlaps with the proximal part of the TRBJ gene, we considered the possibility that residues encoded by TRBJ2-3 might interface with the antigen-HLA-DRB1 complex. Indeed, by aligning CDR3 sequences grouped by length and stratified by J gene usage, we find for lengths 12 and 13 that an aspartate encoded by J2-3 is selected by high-risk HLA-DRB1 complexes (~80% of the TRBJ2-3 clonotypes with CDR3 length 12 or 13, against ~4% of all other TRBJ gene rearrangements)(FIG. 16C). This demonstrates that germline-encoded amino acids from the TRBJ2-3 gene can be found in the CDR3 region and promote enrichment by HLA-DRB1* complexes. Notably, the TRBJ2-3 gene is unique among V and J genes in that its overlap with the CDR3 includes a residue that is independently selected by HLA-DRB1*.

FIG. 16: J2-3 encoded TCRs have CDR3 features preferentially selected by HLA-DRB1 RA risk alleles. (A) Using the association between TCRβ CDR3 features and HLA-DRB1-mediated risk for RA (motif derived from [8]), the CDR3DRB1* risk score was computed for each cell for which single-cell TCR sequencing data was obtained. (B) Among CD4 T cell clones from the blood of RA patients, the CDR3DRB1* selection score is markedly higher in the subset characterized by the TRBJ2-3 rearrangement, suggesting that this subset is particularly enriched in features that are selected by HLA-DRB1 alleles that predispose to RA. (C) For CD4 clones with a specific CDR3 length (12 to left, 13 to right), stratified by TRBJ gene rearrangement (rows), the identity (single-letter amino acid code) and proportion (blue color scale) of clones in each stratum with the most-frequent amino acid at each position (columns). The dark blue boxes in the distal CDR3 region represent amino acids contributed by the germline-encoded TRBJ gene. Beneath each length-specific plot is a motif diagram reflecting the degree of germline-independent association of amino acids at each position with high-risk HLA-DRB1 alleles derived by [8], from which those HLA selected amino acids that are predominant within a single TRBJ-rearranged subset are highlighted by green (for negative selection) or red (for positive selection) boxes. The presence of an HLA-DRB1-selected amino acid overlapping with the TRBJ gene-contributed sequence is unique to TRBJ2-3.

Across 17 individuals with seropositive RA, we isolated CD4+ T cells from peripheral blood and performed paired transcriptomic and targeted TCR single-cell RNA sequencing (FIG. 17A). To assess the degree to which a cell's TCR has features of selection by a high-risk HLA-DRB1* complex, we calculated the CDR3DRB1* selection score [8] for their TCR CDR3 domain (FIG. 16A). For each patient, a mean CDR3DRB1* selection score was then calculated across either total CD4 T cells or sorted naive and non-naïve subsets (~6,500 cells/sample, FIG. 17B, FIG. 28). The HLA-DRB1 alleles for each patient were identified by applying the arcasHLA algorithm [9] and scored for RA risk effect size [8] (FIG. 17B, bottom). Notably, the mean CD4 CDR3DRB1* selection score positively correlated with HLA-DRB1* RA risk scores (FIG. 17C), to a degree qualitatively similar to that found in healthy subjects (slope 0.0042 for healthy subjects [8], slope 0.0032 for RA). For these RA patients, having a single high-risk HLA-DRB1 allele (>1.0 effect score) resulted in a categorical shift in mean CDR3DRB1* scores (−0.01 v. 0.03) These data suggest that the presence of specific HLA-DRB1 alleles, which are known to significantly increase risk of developing RA, impact CD4 T cell selection on a repertoire-wide level in RA patients, similarly to a healthy population. Further, our algorithm can identify these cells in affected patients.

FIG. 17: HLA-DRB1 alleles shape the CD4 TCR repertoire in RA patients. (A) Seventeen donors with RA provided blood samples, comprising approximately 6,500 cells per sample. (B) (upper) The repertoire-wide mean CD4 CDR3DRB1* score for CD4 T cells from RA patient blood samples. (lower) HLA-DRB1 RA risk score for each subject, with gray bars reflecting the combined effect of both HLA-DRB1 alleles on the genetic risk for RA, and the white bars representing the contribution from each allele separately. Patients are sorted first by the magnitude of their single highest-RA-risk HLA-DRB1 allele (tallest white bar), and second by the total HLA-DRB1 RA risk (grey bar). (C) The relationship between the mean CDR3DRB1* score and the HLA-DRB1 RA risk for each of the 26 samples across the 17 RA subjects.

FIG. 21: Bars are colored according to their tissue of origin-blue for synovial CD4 T cells, salmon for total blood CD4 T cells, red for naive blood CD4 T cells, and dark red for non-naive blood CD4 T cells. (A) Number of high-quality barcodes (each representing a cell) obtained for each sample. (B) Number of CD4 T cell clonotypes obtained for each sample. (C) Mean clone size (in number of cells) in each sample. (D) Median clone proportion (as a fraction of the sample total number of cells) for each sample.

Notably, the CDR3DRB1* selection score for J2-3-containing CD4 T cell clones had a positive association with the high-risk HLA-DRB1 alleles in RA patients, whereas J2-6 clones showed no such association (FIG. 18). These data support that the HLA-DRB1 complexes that confer risk of RA disease have a preferential selection, relative to other DRB1 alleles, for CD4 T cells with the TCR encoded by TRB J2-3.

FIG. 18: HLA-DRB1 variants select specific CD4 T cells with J2-3 encoded TCRs in RA. The relationship between CDR3DRB1* selection score and HLA-DRB1-related RA risk is particularly strong in the subset of CD4 T cells with the TRBJ2-3 rearrangement, by contrast with the subset with the TRBJ2-6 rearrangement.

Although subtle, across all RA patients for which we obtained samples of blood and synovium, the frequency of CD4 clonotypes with the TRBJ2-3 rearrangement in synovium was statistically significantly higher than the TRBJ2-3 frequency in blood, uniquely across all TRBJ genes (FIG. 19).

FIG. 19: Increased frequencies of CD4 T cells with TRBJ2-3 TCRs in RA joints.

### Discussion

Previous work has established that variation in HLA-DRB1 is the dominant genetic influence on the risk of developing RA. The mechanism of that relationship is unknown, but based on the role of MHC Class II in presenting antigen to CD4 T cells and the frequent presence of those cells in RA synovium, we and others have been motivated to understand how HLA-DRB1 influences the CD4 T cell repertoire. Here, we have shown that CD4 TCR features preferred by HLA-DRB1 alleles predisposing to RA in healthy individuals are also preferred by HLA-DRB1 alleles in RA patients. We have further identified several unique relationships HLA-DRB1 and germline-encoded TCR features, specifically TRBJ2-3, that are suggestive of a role in RA pathogenesis.

Firstly, we found that TRBJ2-3 is centrally selected by HLA-DRB1 alleles associated with RA. Secondly, TRBJ2-3 clonotypes have on average a markedly higher CDR3DRB1* score, and therefore magnitude of TCR features preferred by RA-predisposing HLA-DRB1 alleles, than any other subset defined by germline-encoded rearrangements. This is largely driven by an aspartate that is with high probability contributed to the CDR3 sequence directly by TRBJ2-3, although we also observe additional indirect TRBJ2-3-specific bias elsewhere in the CDR3.

Finally, although a mild effect, there is a statistically significant increase in the frequency of TRBJ2-3 CD4 clones in the RA synovium relative to blood.

Taken together, these findings are suggestive of a concentration of potentially pathogenic CD4 T cells in the TRBJ2-3 subset. If we consider a model with a local basin of stability reflecting a healthy synovium without active autoimmunity, and a stochastic tendency to push over an activation barrier to a pathogenic autoimmune state that increases as a function of CDR3DRB1* score, we can account for the HLA-DRB1 effect on the propensity for RA intermediated by features of the TCR that are marked by TRBJ2-3. Although this model is not uniquely specified by our available data, it is consistent, and suggests a broader repertoire-wide view of CD4 T cell involvement in RA which motivates therapeutic targeting of the TRBJ2-3 CD4 subset. More broadly, by statistically establishing a connection between HLA-DRB1 and germline-encoded regions of the TCR, our data suggests a model of TCR interaction with the antigen-HLA complex.

### Summary Methods

Nineteen subjects with an RA diagnosis and anti-CCP positivity were chosen from a cohort of RA patients undergoing joint surgery [10] who donated either or both of PBMC and synovial tissue fragments, which were stored in liquid nitrogen. Synovial tissue samples were thawed and disaggregated using a previously optimized protocol [11] and PBMC samples were thawed, from which the CD4+ T cell population was isolated by FACS, with further separation into naive and non-naive CD4+ populations within the PBMC samples from eight of the subjects (detailed staining protocol in Extended Methods). Single cell RNAseq and TCR data were obtained for each sorted population by the 10× Chromium Next GEM Single Cell 5′ cDNA protocol with Human TCR Amplification protocol, with sequencing performed on the Illumina NovaSeq 6000. Sequencing data were processed through the 10× Cellranger (v 7.2.0) pipeline with down sampling for read depth consistency across samples, followed by isolation of high-quality single-cell data using an artificial nearest-neighbors method adapted from [12, 13] and adjustment for batch effects [14, 15]. The combined gene expression and TCR data were processed through a computational pipeline to extract cell identity and clonotype information, including the CDR3 risk score for each clonotype computed as in [8].

### Extended Methods

### Subject Selection and Sample Collection

Subjects were chosen from participants in the RA Perioperative Flare Study [10], which recruits a cohort of patients with rheumatoid arthritis (defined by either or both of the 1987 and/or 2010 ACR criteria for RA) whose immunosuppression was paused in advance of an arthroplasty or joint replacement surgery, under the approval of the Hospital for Special Surgery IRB (#2014-233) with informed consent obtained from all subjects. This clinically well-characterized cohort has contributed to data in multiple studies [16, 17, 18, 19]. For inclusion in this analysis, we selected a subset of this cohort with (i) high-level anti-Ccitrullinated peptide (anti-CCP) positivity, and (ii) synovial tissue demonstrating at least a moderate degree of lymphocyte infiltration as determined by a pathologist specialized in musculoskeletal tissue. For six subjects either a blood or a tissue sample was utilized, for four subjects two tissue samples from distinct joints were utilized, and for twelve subjects paired blood and tissue samples were utilized.

Briefly, according to the RA Perioperative Flare Study protocol, within two hours of surgery, PBMCs from venous blood drawn into heparinized collection tubes were isolated by Ficoll gradient centrifugation using SepMate PBMC isolation tubes (Stemcell Technologies SepMate™-50) and stored at a concentration of 4×10⁶ cells/mL in Cryostor media (Stemcell Technologies CryoStor® CS10) at −196° C. with temperature gradient control (Thermo Scientific Mr. Frosty™Freezing Container), while synovial tissue extracted by the surgeon was physically cut into small fragments and also stored at −196° C. in Cryostor media with a controlled temperature gradient. Samples remained in liquid nitrogen storage until being thawed on the day of FACS sorting and single-cell transcriptomics plus immune receptor repertoire sequencing.

### Single-cell RNAseq and TCR Repertoire Data Acquisition

For synovial tissue samples, after recovery of a single vial (containing approximately six fragments of tissue) from liquid nitrogen and thawing in a dry bead bath at 37° C. for four to six minutes (until only a small amount of frozen sample remained visible), the tissue fragments were rinsed in RPMI, manually mechanically dissociated with dissection scissors, chemically digested with Liberase TL (Roche) and DNAse I at 37° C. under gentle continuous rotation for thirty minutes, further mechanically dissociated by grinding against a 70 μm filter, and lastly rinsed with and resuspended in Ca/Mg-free PBS supplemented with 5% FBS. For stored PBMCs, after thawing a single vial (containing approximately 4 million cells) from liquid nitrogen storage in a dry bead bath at 37° C. for four minutes (until only a small amount of sample remained frozen), the cells were rinsed with RPMI and then rinsed with and resuspended in Ca/Mg-free PBS supplemented with 5% FBS.

After the final post-digestion centrifugation, each sample was resuspended in 50 μL of blocking reagent, transferred to a 96-well plate, and incubated in darkness on ice for ten minutes, after which 50 μL of the fluorescent antibody mix was added and the mixture again incubated in darkness on ice for twenty minutes. Staining was terminated with additional Ca/Mg-free PBS supplemented with 5% FBS, the sample was centrifuged and resuspended twice, and was then transferred to a pre-blocked eppendorf tube. Stained cells were promptly transported to the Weill Cornell Medicine Flow Cytometry Core Facility where they were gated on either CD45(+)/CD3(+)/CD14(-)/CD4(+) (for round 1), CD45(+)CD3(+)/CD14(−)/CD4(+)/CD8(−) (for round 2), CD45(+)CD3(+)/CD14(−)/CD4(+) (for round 3 synovial samples), or CD45(+)CD3(+)/CD14(−)/CD4(+)/CD62(+)/CD45RA(+) and D45(+)CD3(+)/CD14(−)/CD4(+)/(CD45RA−) separately (for round 3 blood samples, to isolate the naive and non-naive peripheral CD4 T cell populations, respectively).

For each sample, the CD4 T cells recovered by FACS were immediately processed through the 10× Chromium Next GEM Single Cell 5′ cDNA protocol (10× Genomics CG000331 Rev E) with the Human TCR Amplification & Library Construction kit protocol, targeting 10,000 cells per sample with 20,000 reads/cell for whole transcriptome and 5,000 reads/cell for TCR profiling. The net effect of this protocol is for each read to be tagged with a barcode uniquely specifying the single cell from which the mRNA originated, although there is an unavoidable technical rate with which a single barcode may label two or more cells. The libraries were pooled to approximately equalize the read depth of each sample and sequenced at the Weill Cornell Medicine Genomics Resources Core Facility on the Illumina NovaSeq 6000 according to the recommended parameters. Read depth was generally at target, with a small number of exceptions where a decreased number of cells led to an increased read depth.

### Single-cell RNAseq Data Processing

Sequencing reads were processed through the 10× Cellranger software (v 7.2.0) with the GRCh38-2020-A reference genome to generate counts per gene for each observed barcode and apply first-pass quality-control filters to isolate barcodes likely to contain well-characterized cell contents. At this stage, before any further filtering on the conventional quality-control metrics (number of genes detected per cell, number of counts per cell, the percentage of counts arising from mitochondrial genes, distributed as in FIG. 20), we removed ‘doublets’—instances where two cells merge with the same barcode-bearing 10× ‘GEM’ in the microfluidic chamber, leading transcript reads from both cells to be inappropriately labeled with the same barcode. FIG. 20: Basic quality control parameters for a single subject. “n_genes_by_counts” is the number of genes observed more than once in each cell, “total_counts” is the total number of counts observed in each cell, and “pct_counts_mt” is the percentage of total counts that originate from mitochondrial genes in each cell. The diagonals are histograms of each QC parameter's distribution, the below-diagonal plots are simple scatter plots for each pair of basic QC values, and the above-diagonal are hexagonal two-dimensional histograms for each pair. To achieve this in the relatively homogeneous CD4 T cell background population we adapted and independently implemented an optimized variant of the the synthetic doublet methods described in [12, 13]. FIG. 22: Example of doublet detection for a single sample. The computed best distribution of the proportion of artificial nearest neighbors (pANN) for each of three choices of doublet generation strategy (whether additive or average, and whether normalized or not) are shown in the lower row, with the actual cells' pANN values in faded blue and the synthetic doublets' pANN values in faded red. It is intuitively clear that the best separation is achieved with additive doublet generation without normalization, and the AUC of the ROC in the upper row plots verifies this. Further supporting the accuracy of this doublet detection, overlaid in solid colors are the pANN distributions of subsets of the observed cells colored according to their number of TCRα and TCRβ chains. Cells with double-TCRα/double-TCRβ chains are colored red, and as expected given the biological improbability of such a state in a single cell (thus very high likelihood of being a doublet) these are far to the right of the pANN distribution, and thus excluded. Double-TCRα/single-TCRβ cells are more biologically plausible, and a greater portion of those (solid blue bars) are retained due to having low pANN, versus those that fall on the high-pANN side of the distribution. Essentially all of the single-TCRα/zero-TCRβ and zero-TCRα/single-TCRβ cells are classified as singlets. Such methods operate on the observation that although we do not initially have a good signal of which barcodes are doublets, if it is true that the substantial majority of barcodes are singlets, for each sample we can generate a large number of ‘synthetic doublets’ by randomly choosing pairs of observed barcodes and combining their reads together. We are then able to calculate which of the observed barcodes have a tendency to appear similar to the artificial doublets based on proximity in the dimension-reduced transcript count space, which is computed as the fraction of neighbors of each actual barcode that are artificial doublets—naturally, if this fraction is high then the actual barcode is likely to itself be a doublet, while if it is low it is likely to be a singlet, and the magnitude of the separation between the higher versus lower observed values of this ‘proportion of artificial nearest neighbors’ (pANN) is a core measure of the quality of the doublet detection. Several free parameters have a significant effect on this degree of pANN separation, such as the number of artificial doublets that are generated, the number of principal components and neighborhood size used to compute the nearest-neighbor distances, and the specifics of how the observed data are combined to generate the artificial doublets (namely, whether simply additive or averaged, and whether or not to normalize the entire sample's transcriptome data; these are points on which the literature does not have a consensus). In light of this ambiguity, we have implemented an efficient, parallelized computation of the pANN that allowed us to avoid having to choose these parameters a priori, and instead chose the best performing option from a wide-ranging scan over possible parameters. In doing this we found that for one specific choice of the synthetic doublet generation (simple additive combination, with no subsequent normalization step), there was consistently a well-separated high-pANN (thus, likely doublet) population of approximately the size expected (FIG. 22), and further that the high-pANN barcodes tend to have an excess of features such as extra TCR chains that, although lacking in specificity to use directly as markers of doublets, would be plausibly expected to occur more often among them.

After discarding barcodes likely to be doublets, transcriptome data was then normalized and log-transformed, and genes only present in very few cells or with low normalized variability were discarded. Transcript counts for each cell were then regressed against the total number of counts and the percentage of counts from mitochondrial genes to minimize their effect on cell subtype identification. Full transcriptional data was reduced to 50-dimensional space with a conventional principal components analysis (PCA). To mitigate batch effects and technical artifacts in each sample, we made use of the Harmony algorithm's Python implementation [14, 15] that iteratively adjusts principal components to minimize features that are more correlated with the effects for which mitigation is sought. As shown by coloring pre-and post-Harmony uniform manifold approximation projection (UMAP) of all cells colored by their sample of origin, it is visibly apparent that Harmony removes some sample-specific effects. The full complement of cells were clustered with the Leiden algorithm [20], with the resolution parameter chosen to generate 13 CD4 subtypes, as that was the number of CD4 subtypes obtained in [11].

### Single-cell TCR Repertoire Data Processing

We employed a series of heuristic measures to improve T cell clonotype characterization by better accounting for unavoidable partial sequencing failures. For instance, if there is a TCR clonotype that is truly (TCRα: x_{α} and TCRβ: x_{β}), we would expect to observe cells with the full pair but also less often (TCRα: Ø and TCRβ: x_{β}) if there were sequencing dropout of TCRα, or (TCRα: x_{α} and TCRβ: Ø) if there were sequencing dropout of TCRβ. Thus, if we were to observe a small number of cells with (TCRα: Ø and TCRβ: x_{β}) or (TCRα: x_{α} and TCRβ: Ø) and a larger number with (TCRα: x_{α} and TCRβ: x_{β}), we should decide that all of those cells are actually members of the same clonotype. Similarly, if there were a TCR clonotype reflecting a failure of a allelic silencing that is truly (TCRα: x_{α1}, x_{α2} and TCRβ: x_{β}), we would similarly expect to see most often the full trio but also less often any two out of the three TCR chains, namely any of (TCRα: Ø, x_{α2} and TCRβ: x_{β}), (TCRα: x_{α1}, Ø and TCRβ: x_{β}), (TCRα: x_{α1}, x_{α2} and TCRβ: Ø). If we were to see smaller numbers of cells with these latter three relative to a larger number of cells with the double-α/single-β TCR, we would decide that all of those cells are members of the same clonotype. In the opposite direction, we also inspected for signatures of a missed doublet, where for instance comparable numbers of (TCRα: x_{α1} and TCRβ: x_{β1}) and (TCRα: x_{α2} and TCRβ: x_{β2}) are observed, with a smaller number of (TCRα: x_{α1}, x_{α2} and TCRβ: x_{β2}) or (TCRα: x_{α1} and TCRβ: x_{β1}, x_{β2}), which would suggest that the double-α or double-β barcodes are in fact doublets, and should be omitted. We systematically extended these heuristics to all accountable single-step error processes, and with this extracted a set of ‘merged’ clonotypes that were used in the downstream analysis.

### TCR Repertoire Diversity and Overlap Metrics

In order to determine to what degree the observed CD4 T cell populations harbor a concentration of clonal expansion in a small subset of clonotypes, a number of diversity metrics have been employed. We have computed two of these, the normalized Shannon entropy and the Gini coefficient. For a T cell repertoire with M distinct clonotypes, and a vector representing the frequency of each clonotype in the repertoire in increasing order (f₁, f₂, . . . , f_{M}), the normalized Shannon entropy is S=Σ^{M}ᵢ₌₁f_{ilog(f}_{ᵢ}₎/log(M). The Gini coefficient of the repertoire is G=(M+1−2*Σ^{M}ᵢ₌₁ Σⁱⱼ₌₁fᵢ/Σ^{M}ᵢ₌₁fᵢ)/M.

For a measure of how similar (or overlapping) two observed CD4 T cell repertoires are, the Horn-Morisita Overlap Index is frequently employed [21, 22]. For two repertoires A and B which between them have M unique clonotypes, for each we have a vector representing the number of cells in each clonotype [formula].

### HLA Risk Score

The arcasHLA algorithm allows extraction of HLA Class II genotypes from scRNAseq data [9]. We used this to infer the HLA types of each of our subjects, and then applied the method of [8] to compute the RA HLA risk score, which is directly related to the log-odds-ratio of RA due to a subject's specific HLA-DRB1 genotype.

FIG. 23: OLS for the relationship between CD4 repertoire mean CDR3 risk and RA HLA risk, computed for all measured samples (top left) and separately for subsets of samples based on their location of origin (blood vs synovium) or identity (naive vs non-naive in blood) FIG. 24: The mean CDR3 risk score (±standard error of the mean) for clonotypes separated by their TRAJ, TRAV, TRBJ, and TRBV rearrangements as labeled, including clonotypes from all samples. The red line is the overall mean across all rearrangements

FIG. 25: Heatmaps of the predominant amino acid (letters superimposed on plot) at each position (horizontal axis) within each TRBJ rearrangement-defined subset of cells (vertical axis), for CDR3 sequences between length 12 and 18. A darker square reflects near-complete dominance at that position of the labeled amino acid, while a lighter square reflects greater diversity at that position. The sequences adjoining each heatmap are the amino acids comprising the proximal end of the corresponding row's TRBJ gene. Outlined boxes indicate CDR3 positions where the predominant amino acid was found to be associated with RA predisposing HLA-DRB1 alleles, with green indicating a negative association (less risky) and red indicating a positive association (more risky).

FIGS. 26 and 27: Heatmaps of the mean CDR3 risk score stratified by individual samples (vertical axis) and TRAJ, TRAV, TRBJ, or TRBV gene rearrangement (horizontal axis).

### Example 3

### HLA Variants Influence TRBV and TRBJ Gene Usage in T Cell Repertoires

To examine if HLA amino acid variation encoded by different alleles influences the selection of specific germline-encoded TCR subunit genes, we analyzed the T cell repertoire of 628 healthy individuals from which bulk TCR sequencing and HLA genotype data were available (FIG. 29A). Focusing on the TCR β subunit (TRB), we computed multidimensional phenotype vectors for each repertoire that represent the frequency of each of the 46 TRBV of 13 TRBJ germline-encoded genes. Using these vectors and the individual's HLA genotype in a multivariate multiple linear regression model (MMLM), we calculated the association of TRBV or TRBJ gene usage with the amino acids at 369 HLA positions that vary across the HLA alleles found this cohort. From this analysis, we found that both TRBV and TRBJ gene usage are significantly associated with HLA amino acid variation, with the strongest associations corresponding to HLA-DRB1 (FIG. 29B-C). These results suggest that the frequency of T cells with specific germline-encoded TCR subunits in an individual's T cell repertoire relates to the HLA gene variants in their genome, particularly variants in the HLA-DRB1 gene.

In regards to the specific residues, we found the strongest association with TRBV gene usage to be with the HLA-DRB1 amino acid 13 (DRB1-13), with additional associations in DRB1-26 and DQB1-86 obtained by serial conditional analyses. For TRBJ gene usage, DRB1-71 was the strongest association, with additional associations in DRB1-74 and DRB1-70. The explained variance of the overall TRBV or TRBJ frequencies by each individual HLA site is shown, with HLA-DRB1 site 13 having the highest explained variance for both TRBJ and TRBV frequencies. Notably, most of the DRB1 positions with TCR germline-encoded gene associations lie within the region of the HLA complex that interface with antigen or TCR. Interestingly, the strongest associations with the TCR β germline-encoded gene usage (HLA-DRB1 amino acids 13, 70, 71, and 74) also comprise the residues that associate with risk of developing RA.

We focused this analysis by determining the association between variation at each HLA site and the TCR repertoire frequencies of each TRBV and TRBJ gene separately using an OLS LM model. We found that among TRBJ genes, the bulk of the effect of HLA variation arises from HLA sites associated with RA (grey background in FIG. 29D), and has the greatest effect on TRBJ2-3 and TRBJ2-5 frequencies. For both TRBJ2-3 and TRBJ2-5, the strongest HLA effect is mediated by DRB1-67, at which alleles with a leucine are known to increase the effective presentation of citrullinated antigen [33]. FIG. 29E shows that the presence of leucine at DRB1-67 increases the TCR repertoire frequency of TRBJ2-3 and TRBJ2-5. Multiple HLA-DRB1 sites associated with RA and other autoimmune diseases were found to be the primary associations with a variety of TRBV genes, suggesting the approach of identifying T cell subsets based on disease HLA genetics may be generalizable to other conditions.

### Example 4

### HLA-DRB1 Alleles Associated With RA Risk Result in Increased TCR β J2-3 and J2-5 Encoded T Cells

Having established that the variation of certain amino acid positions across HLADRB1 alleles influences TCR β V and J gene usage in TCR repertoires, we proceeded to ask if the HLA-DRB1 alleles that associate with RA risk select for T cells with specific V or J genes. Here, rather than examining each HLA-DRB1 allele separately, we generated a combined HLA risk score based on prior knowledge of variation associated with specific disease states. For instance, for RA prior genome-wide association studies and fine-mapping have determined that the HLA DRB1 amino acids at positions 11, 13, 71, and 74 account for most of the risk of disease, and for each haplotype determined by those four amino acids there is an observed odds ratio of developing RA [4]. For each allele in an individual we identify the corresponding haplotype and compute the log odds ratio, and then add together the contribution from each allele, to define the HLA-DRB1 RA risk score of the individual. Using GWAS data for other conditions associated with HLA variation we can similarly compute the disease specific HLA risk score for type 1diabetes (TID, based on HLA-DQB1 position 57 and DRB1 positions 13 and 71) and celiac disease (CD, based on HLA-DRB1 and HLA-DQBI four-digit alleles).

For each individual, we counted the number of T cells encoded by each V and, separately, each J gene and calculated the number of T cells in each group. Comparing these quantities to the RA risk HLA-DRB1 score for each person, we identified two V and two J gene-defined T cell subsets that were increased in individuals with high HLA-DRB1 RA risk scores (V19, V5-8, J2-3 and J2-5), and four V and one J gene defined T cell subsets that were decreased in individuals with high HLA-DRB1 RA risk scores (V15-1, V20-1, V5-6, V14-1) at a Benjamini-Hochberg-corrected p threshold of 0.01. Of the three J gene associations, two (J2-3 and J2-5) were replicated in an independent cohort [34], while out of the six V gene associations, two were replicated.

We performed a similar analysis for two other autoimmune conditions, type 1 diabetes (TID) and celiac disease (CD), using the specific HLA alleles that confer risk for each of these conditions. This analysis demonstrated positive associations for four V gene T cell subsets for TID and five V gene subsets for CD with the corresponding HLA risk alleles for each condition. Thus, across the three disorders, only two J gene T cell subtypes associated with disease risk; the J2-3 and J2-5 associations found in RA (top-right stars in FIG. 30B).

### Example 5

### Selection in the CDR3 Region of TCR β J2-3 T Cells by HLA-DRB1 RA Risk Alleles in RA Patients

We next looked to address if the germline-encoded subsets of the TCR repertoire which are increased in individuals with high-RA-risk HLA-DRB1 alleles (TCR 8 V19, V12-3/4 and J2-3) relate to features found in the TCR that interface with the HLA-antigen pocket (the CDR3region). To do this, we assessed for TCR 3 CDR3 features that were recently shown to be selected by HLA-DRB1 RA high-risk alleles (FIG. 31A)[8]. Scoring for these CDR3 features across V and J gene T cell subsets, TRBJ2-3-encoded T cells demonstrated the highest mean score (p<0.05 nominal, with Bonferroni correction) (FIG. 31B), suggesting that J2-3 can encode for features preferred by the HLA-DRB1 high-risk alleles. More specifically, by aligning the CDR3 sequences by length, we found for CDR3 sequences of lengths 12 and 13 that J2-3 often encodes for an aspartate that is significantly selected by high-risk HLA-DRB1* alleles (present in 80% of the TRBJ2-3 clonotypes of these lengths, versus 4% of all other TRBJ genes) (FIG. 31C). This was the only V or J germline-encoded amino acid that demonstrated strong positive association across any CDR3 length (FIG. 31C), whereas several amino acids from various V and J genes displayed negative associations (FIG. 31C).

Having found that TRBJ2-3 can encode CDR3 features selected by HLA-DRB1 RA risk alleles, we next tested if those features are involved in the selection by high risk HLA alleles in RA patients. To do this, we isolated CD 4+T cells from peripheral blood of 17 individuals with RA, performed paired single-cell transcriptome and TCR RNA sequencing, and calculated the mean CDR3DRB1* selection score across total or naive and non-naive CD4 T cell subsets (FIG. 31E y-axis, ~6,500 cells/sample). The HLA-DRB1 alleles found in each patient were identified by applying the arcasHLA algorithm to the single-cell transcriptomic data [9] and then scored for RA risk as described above (FIG. 31E, -axis). Notably, as previously shown in a healthy cohort, the mean CDR3DRB1* selection score for CD4 T cells from RA patients positively correlated with HLA-DRB1* RA risk score, suggesting that the alleles known to increase the risk of RA influence the selection of CD4 T cells with the specific CDR3 features that contribute to the CDR3DRB1* selection score.

However, this association is not uniformly seen in all subsets of T cells. Strikingly, the influence of HLA-DRB1* on TCR repertoire CDR3 features is restricted to the TRBJ2-3 subset. Looking at V and J-gene T cell subsets, we found that the J2-3 encoded T cells in RA patients with high risk HLA-DRB1 alleles not only have higher scores for these CDR3 features than RA patients without these HLA alleles, but also that the relationship between HLA alleles and CDR3 features is statistically significant only in the J2-3 subset (FIG. 31F-G). This restriction suggests that the mechanisms for HLA-DRB1* selection of TCR J2-3 and the CDR3 features identified in are linked. Furthermore, this finding is unaffected by removing single CDR3 motif features from the calculation, telling us that this finding is not due to a singular CDR3 feature but is due to the composite motif effect.

### Example 6

### The RA Synovium Favors TRBJ2-3 CD4 T Cells

As the primary site of immune-mediated damage in RA is the synovium, we additionally obtained scRNAseq and scTCRseq data for CD4 T cells from synovial tissue samples obtained surgically from thirteen of the 17 RA patients whose blood was analyzed above. Within this subset of patients, three had undergone two orthopedic operations on different joints, allowing us to separately analyze the CD4 T cell repertoires in each of a pair of joints within the same individual. In each of those multi-synovium patients, we found a greater degree of overlap between the CD4 clones found in the two joints than between either joint and the blood compartment (FIG. 32A).

In light of this evidence suggesting that specific CD4 T cell clones are preferentially attracted to and/or retained in the joints of RA patients, we further sought to determine if there might also be a tendency for specific germline-encoded sequence subsets of CD4 T cells to occupy the diseased joint. In our data, the frequency of CD4 clonotypes with the TRBJ2-3 rearrangement in synovium was statistically significantly higher than the TRBJ2-3 frequency in blood, uniquely across all TRBJ genes (FIG. 32B). Utilizing a generalized linear mixed model (GLMM) to more robustly quantify this effect by exploiting the single-cell structure of our repertoire data, by conditioning on subject identity we are able to discern a very high statistical significance of this effect (FIG. 32C). This suggests that T cells with TRBJ2-3, relative to other subsets, have a selective advantage for occupying the synovial tissue of RA patients.

### Example 7

### Discussion

Previous work has established that variation in HLA-DRB1 is the dominant genetic influence on the risk of developing RA. The mechanism of that relationship is unknown, but based on the role of MHC Class II in presenting antigen to CD4 T cells, the importance of the overall TCR repertoire in determining activation by specific antigens, and the frequent presence of T cells in RA synovium, we and others have been motivated to understand how HLA-DRB1 influences the CD4 T cell repertoire. Here we have shown that HLA variation at residues implicated in RA development by GWAS influences the usage of germline-encoded V and J gene sequences in the TCR repertoire, with a specific connection between RA-associated HLA-DRB1 alleles and usage of the TRBJ2-3 gene in the TCR. We have further identified unique relationships between HLA-DRB1* alleles and germline-encoded TCR features, specifically TRBJ2-3, that are suggestive of a role in RA.

Firstly, we found that the same positions in HLA-DRB1 where variation modulates risk for RA also most strongly shape the T cell repertoire proportions of the germline-encoded TRBV and TRBJ regions of the TCR. We also analyzed a crystal structure of the HLA-antigen-TCR complex that suggests a direct interaction between HLA-DRB1 and the TRBJ region of the TCR. Secondly, we show that TRBJ2-3 is centrally selected by HLA-DRB1 alleles associated with RA, uniquely among TRBJ gene-defined T cell subsets. Thirdly, TRBJ2-3 clonotypes have on average a markedly higher CDR3DRB1* score, and therefore magnitude of TCR CDR3 features preferred by RA-predisposing HLA-DRB1* alleles, than any other subset defined by germline-encoded rearrangements. This is largely driven by an aspartate that is with high probability contributed to the CDR3 sequence directly by TRBJ2-3, although we also observe additional indirect TRBJ2-3-specific bias elsewhere in the CDR3. Fourth, the HLA-DRB1* selection of CDR3 features in our panel of RA patients is uniquely significant in the TRBJ2-3 subset, suggesting a restriction of the HLA-antigen effect on CDR3 to one specific germline-encoded subset. Finally, there is a statistically significant increase in the frequency of TRBJ2-3 CD4 clones in the RA synovium relative to blood.

Taken together, these findings suggest an aspect of TCR recognition that is not purely driven by the interaction between the TCR CDR3 and antigen, but additionally depends on (i) the HLA complex within which antigen is presented to T cells and (ii) the germline-encoded regions of the TCR, which may then be permissive for a ‘fine-tuning’of antigen recognition by CDR3.

This is particularly germane in conditions for which evidence suggests a T cell-mediated mechanism but there is no singular autoantigen that has been identified. Rather, disease-associated HLA alleles may lead to a T cell repertoire with imbalanced representation of specific germline-encoded regions, and those CD4 T cells as a group may then have a tendency to recognize classes of self-antigen that localize to a specific compartment where disease becomes manifest. Specifically for RA, in which a singular autoantigen has not been identified, we have shown that disease-associated HLA-DRB1 alleles increase the representation of TRBJ2-3-containing T cells within the larger repertoire, and that the same TRBJ2-3 subset is the one in which HLA selection of the CDR3 region is most potent. This suggests a mechanism in which HLA interaction with the germline-encoded regions of the TCR promotes a partial stabilization of the overall TCR-antigen-HLA complex that then enables CDR3-antigen interaction. This is reminiscent of the concept recently proposed in the context of antibody recognition wherein germline-encoded GRAB motifs predispose antibodies to recognize specific antigenic structures [20 ]. Such a model would account partially for the non-deterministic relationship between RA genetics and disease by the stochasticity in the magnitude of TCR germline-encoded region selection.

The enrichment of the same TRBJ2-3 subset in the synovium relative to the blood in subjects with RA suggests a tendency of that subset to recognize self-antigen within the joint.

Interestingly, a similar enrichment of TRBV20-1 containing T cell clonotypes in synovium is also observed, in contrast with negative selection of the TRBV20-1 subset by RA-related HLA-DRB1alleles, potentially suggestive of a role for the TRBV20-1 subset in attempting to restore homeostasis in a diseased joint. The combination of direct HLA-DRB1 selection for TRBJ2-3 CD4 T cells in RA, HLA-DRB1 selection for CDR3 features in the TRBJ2-3 subset, and preference of the TRBJ2-3 subset for the synovium suggests that targeting the TRBJ2-3 subset of T cells by means of an antibody or siRNA-mediated treatment may represent a more precisely targeted treatment or prevention strategy in RA. We have also found that HLA variation at sites associated with multiple non-RA autoimmune diseases influences specific TCR repertoire germline-encoded sequence features, providing a framework for extending our approach of exploiting known HLA/disease relationships to identify TCR repertoire features of potentially pathogenic interest in other conditions.

### REFERENCES

Axel Finckh et al. “Global epidemiology of rheumatoid arthritis”. In: Nature Reviews. Rheumatology 18.10 (October 2022), pp. 591-602. issn: 1759-4804. doi: 10.1038/s 41584-022-00827-y.

Josef S. Smolen, Daniel Aletaha, and Iain B. McInnes. “Rheumatoid arthritis”. In: Lancet (London, England) 388.10055 (October 2016), pp. 2023-2038. issn: 1474-547X. doi: 10.1016/S0140-6736(16) 30173-8.

J. L. Newton et al. “A review of the MHC genetics of rheumatoid arthritis”. In: Genes and Immunity 5.3 (May 2004). issn: 1466-4879. doi: 10.1038/sj.gene.6364045.

Soumya Raychaudhuri et al. “Five amino acids in three HLA proteins explain most of the association between MHC and seropositive rheumatoid arthritis”. In: Nature Genetics 44.3 (January 2012), pp. 291-296. issn: 1546-1718. doi: 10.1038/ng.1076.

M. M. Davis and P. J. Bjorkman. “T-cell antigen receptor genes and T-cell recognition”. In: Nature 334.6181 (August 1988), pp. 395-402. issn: 0028-0836. doi: 10.1038/334395a0.

T. P. Arstila et al. “A direct estimate of the human alphabeta T cell receptor diversity”. In: Science (New York, N. Y.) 286.5441 (October 1999), pp. 958-961. issn: 0036-8075. doi: 10.1126/science.286.5441.958.

Ryan O. Emerson et al. “Immunosequencing identifies signatures of cytomegalovirus exposure history and HLA-mediated effects on the T cell repertoire”. In: Nature Genetics 49.5 (May 2017). Publisher: Nature Publishing Group, pp. 659-665. issn: 1546-1718. doi: 10.1038/ng.3822. (Visited on 03/06/2024).

Kazuyoshi Ishigaki et al. “HLA autoimmune risk alleles restrict the hypervariable region of T cell receptors”. In: Nature Genetics 54.4 (April 2022), pp. 393-402. issn: 1546-1718. doi: 10.1038/s41588-022-01032-z.

Rose Orenbuch et al. “arcasHLA: high-resolution HLA typing from RNAseq”. In: Bioinformatics (Oxford, England) 36.1 (January 2020), pp. 33-40. issn: 1367-4811. doi: 10.1093/bioinformatics/btz474.

Susan M. Goodman et al. “Flares in Patients with Rheumatoid Arthritis after Total Hip and Total Knee Arthroplasty: Rates, Characteristics, and Risk Factors”. In: The Journal of Rheumatology 45.5 (May 2018), pp. 604-611. issn: 0315-162X. doi: 10.3899/jrheum.170366.

Fan Zhang et al. “Deconstruction of rheumatoid arthritis synovium defines inflammatory subtypes”. In: Nature 623.7987 (November 2023), pp. 616-624. issn: 1476-4687. doi: 10.1038/s41586-023-06708-y.

Samuel L. Wolock, Romain Lopez, and Allon M. Klein. “Scrublet: Computational Identification of Cell Doublets in Single-Cell Transcriptomic Data”. In: Cell Systems 8.4 (April 2019). Publisher: Elsevier, 281-291.e9. issn: 2405-4712. doi: 10.1016/j.cels.2018.11.005. url: https://www.cell.com/cell-systems/abstract/S2405-4712(18)30474-5 (visited on Apr. 29, 2024).

Christopher S. McGinnis, Lyndsay M. Murrow, and Zev J. Gartner. “DoubletFinder: Doublet Detection in Single-Cell RNA Sequencing Data Using Artificial Nearest Neighbors”. In: Cell Systems 8.4 (April 2019). Publisher: Elsevier, 329-337.e4. issn: 2405-4712. doi: 10.1016/j.cels.2019.03.003.

Ilya Korsunsky et al. “Fast, sensitive and accurate integration of single-cell data with Harmony”. In: Nature Methods 16.12 (December 2019), pp. 1289-1296. issn: 1548-7105. doi: 10.1038/s41592-019-0619-0.

Kamil Slowikowski, John Arevalo, and Jonathan Manning. slowkow/harmonypy: harmonypy version 0.0.9. November 2022. doi: 10.5281/zenodo.7351719. url: https://zenodo.org/records/7351719 (visited on Apr. 30, 2024).

William Stephenson et al. “Single-cell RNA-seq of rheumatoid arthritis synovial tissue using low-cost microfluidic instrumentation”. In: Nature Communications 9 (Feb. 23, 2018), p. 791. issn: 2041-1723. doi: 10.1038/s41467-017-02659-x. url: https://www.ncbi.nlm.nih.gov/pmc/articles/ PMC5824814/.

Dana E. Orange et al. “Identification of Three Rheumatoid Arthritis Disease Subtypes by Machine Learning Integration of Synovial Histologic Features and RNA Sequencing Data”. In: Arthritis & Rheumatology (Hoboken, N.J.) 70.5 (May 2018), pp. 690-701. issn: 2326-5205. doi: 10.1002/art.40428.

Susan M. Goodman et al. “Rheumatoid Arthritis Flares After Total Hip and Total Knee Arthroplasty: Outcomes at One Year”. In: Arthritis Care & Research 72.7 (July 2020), pp. 925-932. issn: 2151-4658. doi: 10.1002/acr.24091.

Dana E. Orange et al. “Histologic and Transcriptional Evidence of Subclinical Synovial Inflammation in Patients With Rheumatoid Arthritis in Clinical Remission”. In: Arthritis & Rheumatology (Hoboken, N.J.) 71.7 (July 2019), pp. 1034-1041. issn: 2326-5205. doi: 10.1002/art.40878.

V. A. Traag, L. Waltman, and N. J. van Eck. “From Louvain to Leiden: guaranteeing well-connected communities”. In: Scientific Reports 9.1 (March 2019), p. 5233. issn: 2045-2322. doi: 10.1038/s41598-019-41695-z.

Tricia Cottrell et al. “Evaluating T-cell cross-reactivity between tumors and immune-related adverse events with TCR sequencing: pitfalls in interpretations of functional relevance”. In: Journal for Immunotherapy of Cancer 9.7 (July 2021), e002642. issn: 2051-1426. doi: 10.1136/jitc-2021-002642. url: https://www.ncbi.nlm.nih.gov/pmc/articles/PMC8261872/ (visited on Dec. 2, 2023).

Henk Wolda. “Similarity indices, sample size and diversity”. In: Oecologia 50.3 (September 1981), pp. 296-302. issn: 1432-1939. doi: 10.1007/BF 00344966.

Falk, K., Rotzschke, O., Stevanovic, S., Jung, G. & Rammensee, H. G. Allele specific motifs revealed by sequencing of self-peptides eluted from MHC molecules. Nature 351, 290-296 (1991).

Miles, K. M., Miles, J. J., Madura, F., Sewell, A. K. & Cole, D. K. Real time detection of peptide-MHC dissociation reveals that improvement of primary MHC-binding residues can have a minimal, or no, effect on stability. Molecular Immunology 48, 728-732 (2011). URL https://www.ncbi.nlm.nih.gov/pmc/articles/PMC3032881/.

Xia, H. et al. Computational prediction of MHC anchor locations guides neoantigen identification and prioritization. Science immunology 8, eabg2200 (2023).

Dunlap, G. et al. Clonal associations between lymphocyte subsets and functional states in rheumatoid arthritis synovium. Nature Communications 15, 4991 (2024).

Rojo, J. M., Bello, R. & Portol'es, P. T-cell receptor. Advances in Experimental Medicine and Biology 640, 1-11 (2008).

Laydon, D. J., Bangham, C. R. M. & Asquith, B. Estimating T-cell repertoire diversity: limitations of classical estimators and a new approach. Philosophical Transactions of the Royal Society of London. Series B, Biological Sciences 370, 20140291 (2015).

Lefranc, M.-P. et al. IMGT unique numbering for immunoglobulin and T cell receptor variable domains and Ig superfamily V-like domains. Developmental and Comparative Immunology 27, 55-77 (2003).

Sharon, E. et al. Genetic variation in MHC proteins is associated with T cell receptor expression biases. Nature Genetics 48, 995-1002 (2016).

Gao, K. et al. Germline-Encoded TCR-MHC Contacts Promote TCR V Gene Bias in Umbilical Cord Blood T Cell Repertoire. Frontiers in Immunology 10, 2064 (2019).

Russell, M. L. et al. Combining genotypes and T cell receptor distributions to infer genetic loci determining V(D)J recombination probabilities. eLife 11, e73475 (2022).

Freed, B. M., Schuyler, R. P. & Aubrey, M. T. Association of the HLA-DRB1 epitope LA67, 74 with rheumatoid arthritis and citrullinated vimentin binding. Arthritis & Rheumatism 63, 3733-3739 (2011). URL https://onlinelibrary.wiley.com/doi/abs/10.1002/art.30636. eprint: https://acrjournals.onlinelibrary.wiley.com/doi/pdf/10.1002/art.30636.

Ota, M. et al. Dynamic landscape of immune cell-specific gene regulation in immune-mediated diseases. Cell 184, 3006-3021.e17 (2021).

Shrock, E. L. et al. Germline-encoded amino acid-binding motifs drive immunodominant public antibody responses. Science (New York, N.Y.) 380, eadc9498 (2023).

Slowikowski, K. harmonypy: A data integration algorithm. URL https://github.com/slowkow/harmonypy.

All documents cited in this specification are incorporated herein by reference are incorporated by reference. US Provisional Patent Application No. 63/721,672 is incorporated herein by reference in its entirety. While the invention has been described with reference to particular embodiments, it will be appreciated that modifications can be made without departing from the spirit of the invention. Such modifications are intended to fall within the scope of the appended claims.

1. A method for treating rheumatoid arthritis (RA), the method comprising administering to the subject a therapeutically effective amount of a compound that targets and/or depletes CD4 positive (+) T cells comprising T cell receptor beta joining 2-3 (TRBJ2-3), T cell receptor beta variable 19 (TRBV19), T cell receptor beta variable 20-1 (TRBV20-1), or T cell receptor beta variable 15-1 (TRBV2-1).

2. The method of claim 1, wherein the compound depletes CD4+ T cells comprising TRBJ2-3.

3. The method of claim 1, wherein the compound is an immunoglobulin, optionally an antibody or an antibody fragment, optionally a monoclonal antibody, optionally a humanized monoclonal antibody.

4. The method of claim 1, wherein the compound is an immunoglobulin that targets TRBJ2-3.

5. The method of claim 1, wherein the compound is an immunoglobulin that targets C-terminal aspartic acid (D) amino acid of a complementarity-determining region 3 (CDR3) of the T cell receptor of the T cell.

6. The method of claim 1, wherein the compound is administered systemically, optionally intravenously.

7. The method of claim 1, wherein the compound is administered via a synovial injection.

8. The method of claim 1, wherein the compound is an antisense oligonucleotide (ASO), RNAi, small interfering RNA (siRNA), or microRNA (miRNA).
